(19) United States
(12) Patent Application Publication
(10) Pub. No.: US 2007/0248487 A1
(43) Pub. Date: Oct. 25, 2007

(54) SYSTEMS AND PROCESSES FOR DISINFECTING LIQUIDS

(76) Inventors: Robert E. Kay, West Chester, PA (US); Victor N. Ballard, West Chester, PA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/691,784

(22) Filed: Mar. 27, 2007

Related U.S. Application Data
(60) Provisional application No. 60/786,523, filed on Mar. 27, 2006.
(60) Provisional application No. 60/790,087, filed on Apr. 7, 2006.

(51) Int. Cl. A61L 2/10; B01J 19/08; B01J 19/12

(52) U.S. Cl. 422/24; 422/186.04; 422/186.3

(57) ABSTRACT

Systems and processes for disinfecting fluids, such as water, use a mercury-free source of ultraviolet radiation such as a flash-lamp. The systems and processes can be used, for example, to inactivate pathogens such as bacteria, spores, and viruses, and pyrogens such as endotoxin in the fluids.

### CROSS-REFERENCE TO RELATED APPLICATIONS

This application claims priority under 35 U.S.C. § 119(e) to U.S. provisional application No. 60/786,523, filed Mar. 27, 2006, and U.S. provisional application No. 60/790,087, filed Apr. 7, 2006. The contents of each of these applications is incorporated by reference herein in its entirety.

### TECHNICAL FIELD

The present invention relates to systems and methods for disinfecting fluids such as water, by inactivating pathogens such as bacteria, spores, and viruses, and pyrogens such as endotoxin in the fluids using ultraviolet light.

### BACKGROUND

There are a substantial number of applications that require the instantaneous disinfection of a fluid that flows at irregular intervals such as hotels overseas, homes and offices without public water and appliances such as refrigerators and soda fountains. Even for those regions with good public water, a safeguard at points-of-use is required to mitigate against failures of the water treatment infrastructure or bio-terrorist attack.

Currently, organizations such as the National Sanitation Foundation are considering raising the required ultraviolet (UV) disinfection dose from 40 mJ/cm² to 259 mJ/cm² due to a recommendation by the Environmental Protection Agency to provide a 4-log reduction of Adenovirus. Conforming to this specification would require that the power of many existing systems be increased by 650% throughout the infrastructures. It would be more efficient to disinfect to such high levels only the water that will be used or consumed at points-of-use. The energy and cost savings from intermittent operation would be significant and a revision of the infrastructure could be avoided.

Even continuously-operated, high-capacity water purification systems, such as those used in pharmaceutical and medical industries, that utilize conventional UV systems can benefit from intermittently operated UV disinfection systems at points-of-use. Such systems generally purify a large volume of water and it is difficult to achieve extremely high levels of microbial inactivation and endotoxin reduction at high flow rates. A device that could instantaneously disinfect water and eliminate exdotoxin could produce, at a point-of-use, water-for-injection (WFI) standard water from United States Pharmacopoeia (USP) or lower quality water. The commercialization of such a device would be beneficial to the Bio-pharmaceutical industries, especially for the processing of injectables, and for the medical industries for applications such as dialysis.

With regard to pathogen content, the USP informational section recommends an action limit of 10 cfu/100 mL, but does not require a specific limit for endotoxins. The USP monograph does not require lower concentrations of bacteria for WFI and WFI water does not need to be sterile. However, the USP monograph does require that WFI water not contain more than 0.25 endotoxin units (EU) per mL. Endotoxins are a class of pyrogens that are components of the cell wall of gram-negative bacteria (the most common type of bacteria in water) and are shed during bacterial cell growth and from dead bacteria. Therefore, WFI water must be of exceptionally high microbial quality in order to have a low endotoxin concentration.

Consequently, WFI water systems are generally more expensive to construct and maintain than USP systems because they require more capital equipment, to provide a higher level of purification, and the water distribution loops must be hot-water-sanitized more often, requiring down-time and vast amounts of energy. A device that could inactivate pathogens and endotoxins at the instant that a point-of-use valve was activated would not only enable facilities with USP quality water to meet WFI requirements, to expand into injectable applications for example, but would also mitigate against intermittent problems inherent to all large-scale high-purity water purification and distribution systems. An instantly acting point-of-use device would serve as an insurance policy for all types of USP water systems and would be invaluable in that pathogen testing requires up to 48 hours and any drugs processed subsequently to a failed water sample cannot be used. A point-of-use device utilized in this capacity would provide the best solution because most problems are caused by bacterial growth downstream of the water purification equipment such as bio-film in the water distribution system. The widespread use of such devices could potentially save millions of dollars for a Pharmaceutical facility.

Additionally, a miniaturized and efficient UV disinfection system with high power density and a rugged lamp that does not contain toxic mercury, as conventional technology utilizes, is particularly well suited for portable operation by travelers overseas, recreational users, the military and emergency response organizations.

Existing ultraviolet light (UV) sterilization systems typically utilize mercury vapor lamps to produce germicidal radiation for the purpose of inactivating microorganisms. Large-scale mercury vapor lamps can be very efficient, converting up to about 30-40% of input electrical energy to a narrow band of radiation at 254 nanometers (nm) that closely matches the maximum absorption of DNA at 260 nm. For this reason, mercury vapor lamps are widely used for disinfection.

However, the output of radiation from a mercury vapor lamp is adversely affected by temperature because the mercury vapor lamp must heat up to specific temperatures before some or all of the liquid mercury will vaporize. It is the concentration of mercury atoms suspended in a gas that produces the germicidal radiation when excited by an electrical current. Because mercury is a liquid at room temperature, mercury vapor lamps require a warm-up period of up to several minutes before reaching maximum irradiance.

Conversely, the efficiency of the mercury vapor lamp will decrease as the lamp exceeds the optimum temperature and the vapor pressure rises to a point where the kinetic energies of the electrons are reduced by more frequent collisions with the higher concentrations of atoms, resulting in the production of lower energy UV photons outside of the germicidal region of 175-320 nm and re-absorption of the emitted UV photons by mercury atoms in resonance. For example, some mercury vapor lamps can lose about 25 percent of their efficiency when diverging from their operating temperature by about 10° C. The operating temperature of conventional UV disinfection systems utilizing mercury vapor lamps can be affected by the fluid media being treated in that a lamp may cool when the process fluid is flowing or may heat up when the fluid is stagnant. For this reason, the efficiency of a conventional disinfection system can be adversely affected by intermittently flowing fluids.

Additionally, the filaments of a mercury vapor lamp are delicate and can be damaged by repetitively cycling the lamp on and off. Because of these limitations, most conventional fluid disinfection systems are operated continuously, although methods have been developed to provide for the disinfection of intermittently flowing fluids with limited success.

U.S. Pat. No. 4,464,336, the contents of which is incorporated by reference herein in its entirety, discloses the use of a flash discharge ultraviolet lamp for disinfection. Systems utilizing flash-lamp technology, sometimes referred to as pulsed-UV (PUV), for disinfection have been developed. For example, U.S. Pat. No. 4,464,336, the contents of which is incorporated by reference herein in its entirety, discloses the use of a flash discharge ultraviolet lamp for disinfection. PUV-based, i.e., flash-lamp, disinfection systems, in general, have achieved limited commercial success due to their high cost of construction and improper modes of operation.

For example, liquid cooling is required for flash-lamps with a wall loading exceeding 30 W/cm², to extend the life of the lamp by preventing catastrophic failure, reducing vaporization of the inside of the quartz lamp envelope and sputtering of the electrodes. Many conventional PUV systems utilize water-cooling systems originally developed by the laser industries. Such systems typically include a pump, a fluid reservoir, and a re-circulating water loop that flows fluid between the flash-lamp and a liquid-tight quartz sleeve. Intensive in-line filtration and de-ionization components are usually required in such cooling systems to provide a clear distillate. Clear distillate is necessary to prevent attenuation of the UV radiation and to prevent a short across the lamp or corrosion of the electrodes.

Additionally, conventional PUV disinfection systems commonly utilize a trigger method, known to those skilled in the art as a ‘simmer,’ in which the lamp is constantly ionized by a direct current (DC) source. The purpose of this method is to improve lamp life by reducing sputtering of the tungsten cathode of the lamp by heating the cathode, and to center the arc within the lamp envelope. Starting the simmer circuit in water requires a sizeable series trigger transformer, because the secondary winding of the transformer is usually connected in series with the main discharge circuit. Consequently, the secondary winding carries thousands of peak amps and has a high turns ratio to the primary winding, for the purpose of generating an output of up to tens of thousands of volts from an input of several hundred volts. Initiating each pulse requires an expensive and substantial switch, such as a thyristor, MOSFET or IGBT, that is capable of holding off several thousand volts from the main discharge capacitor and delivering several thousand amps during the peak of the discharge.

Because of the need for a cooling system, simmer circuit, and other specialized components, conventional PUV disinfection systems are often bulky and expensive to construct, and are not well suited for use as small-scale, personal-use type water disinfection systems. Consequently, companies that provide such systems must usually target markets, such as municipal wastewater, that utilize competitive technology in the form of large and expensive conventional mercury UV disinfection systems. Although PUV disinfection systems typically have greater power density than low-pressure mercury vapor lamps, and unique benefits of PUV-based disinfection have been demonstrated, PUV disinfection systems usually cannot compete with the medium pressure mercury vapor lamps used in large-scale conventional systems.

For example, medium pressure (MP) mercury UV systems widely used in municipal wastewater treatment are about 10% to about 20% efficient. Such MP lamps have a germicidal UV output of about 5 W/cm to about 30 W/cm. The recommended average input power for a flash-lamp utilizing liquid cooling is about 30 W/cm² to about 200 W/cm² with about 240 W/cm² being the maximum for a highly UV transparent lamp like those used for PUV. It is widely accepted that flash-lamps can convert between about 50% to about 60% of input energy to radiation. About 50% photometric efficiency would be optimistic for a PUV system with any resistance, such as a semiconductor switch as required by the simmer circuit, in the discharge path because a considerable percentage of input energy will be dissipated as heat. For example, for a substantial 7 mm bore Suprasil™ lamp operated at the maximum power density, the required germicidal UV content in all radiation required to compete with a MP lamp would be as follows.

$$\frac{30 W \text{/} cm}{240 W \text{/} {cm}^{2} \times π \times .7 cm \times 50 \%} = 11.4 \%$$

As discussed in U.S. Pat. No. 6,228,332, the contents of which is incorporated by reference herein in its entirety, at least about 5 percent, and preferably at least about 10 percent of the energy of the light pulses will be at wavelengths shorter than 300 nanometers. Such systems may fall short when competing with conventional MP mercury UV technology, even when fully optimized and operated at the lamp's highest power density. Additionally, the UV efficiency of a PUV system that can rival MP mercury UV technology is about 5.7% (11.4%×50%) compared to the about 10% to about 20% for MP mercury UV lamps. Therefore, the conventional PUV system will consume two to four times more power.

The efficiency of such a PUV disinfection system utilizing conventional cooling systems and a simmer is also substantially reduced because the power required to operate the water cooling pumps and the energy to maintain the simmer, in the hundreds of volts and up to several amps, consumes many hundreds of watts of power that must be taken into account when estimating germicidal efficiency, which is the amount of germicidal UV energy generated from total input power.

Evaluated in this way, the germicidal efficiency of such conventional PUV systems utilizing simmer circuits is reduced as the pulse rate of the flash-lamp is decreased. Consequently, although simmers are employed to increase lamp life, such conventional trigger methods actually increase the frequency of lamp replacements. This can be illustrated by the parameters denoted in U.S. Pat. No. 6,054,097, the contents of which is incorporated by reference herein in its entirety, as summarized below.
Cap: 20 uF, 3,600V Rep Rate: up to 30 Hz Simmer: 140V×3 A=420 W

$$\frac{20 {E}^{- 6} \times {3600}^{2}}{2} = 130 J per \text{-} pulse \times 30 Hz = \\ 3 , 900 W Peak \frac{420 W}{3900 W + 420 W} = 10 \%$$

The above-noted conventional system is losing about 10% of the input energy to the simmer at the maximum rep rate of 30 Hz. The system will loose 14% at 20 Hz, 24% at 10 Hz, 61% at 5 Hz, until the system eventually looses 76% of input energy at 1 Hz. In order to maintain germicidal efficiency with a simmer, the system therefore must be operated at a high frequency. Because a flash-lamp's life is rated as a number of shots, a higher frequency operation will have the opposite effect of prolonging the maintenance period because it requires that the lamp be replaced more often.

Additionally, conventional PUV disinfection systems may require many thousands of volts to produce substantial UV light because they have a dynamic resistance characteristic, known to those skilled in the art as Kₒ, of between 35 and 70 Ohm Ampres^{1/2}, or higher. For example, U.S. Pat. No. 6,200,466, the contents of which is incorporated by reference herein in its entirety, discloses the following parameters.
l—Lamp Length (cm): 33.5 d—Lamp Bore (cm): 1 P—Fill Pressure (torr): 760

$$Estimated {K}_{o} ( Ω {A}^{1 / 2} ) = 1.27 ( \frac{33.5}{1} ) {( \frac{760}{450} )}^{1 / 5} = 47.2 Ohm {A}^{1 / 2}$$

High voltage is necessary to make efficient UV with lamps with Kₒ values within the prior art because the plasma temperature of the discharge and the corresponding blackbody radiation profile of the lamp is mainly influenced by current density. To reach a current density of about 6,500 A/cm², a minimum regime required to make efficient UV light with the prior art lamp, the required current is as follows.

$$6 , 500 A \text{/} {cm}^{2} \times π \times {( \frac{1 cm}{2} )}^{2} = 5 , 105 A$$

Subsequently, the voltage required is ±47.2|5,105|^{1/2}=3,372 V.

This operating voltage requires large and specialized components imported from the optoelectronic and other specialized industries, again contributing to the bulk and expense of such prior art PUV systems. Although the authors of this patent and others mention systems that are suitable for home use, evaluation of the Kₒ values of all prior art systems clearly illustrate that the embodiments were not intended for small-scale applications and would be too expensive and the voltages involved too dangerous for individual use.

Additionally, conventional PUV disinfection systems typically do not address the production of small-scale PUV water disinfection systems in the designs of their reactor vessels. The ultra-high intensity and power density of a PUV lamp can be problematic because the reactor vessel length is shorter than in systems using conventional mercury vapor lamps, which are relatively long in length. Volumes become smaller and residence-times decrease, contributing to hydraulic problems such as short-circuits in the fluid.

For example, U.S. Pat. No. 6,200,466, the contents of which is incorporated by reference herein in its entirety, teaches the use of a vessel that is larger in diameter than it is long. This expansion of the vessel diameter creates more residence time, but also creates a region of distance-attenuated UV light along the inner surface of the reactor vessel wall. Pathogens traveling through this region may receive an ineffective dose of UV light. Baffles are sometimes used to repeatedly redirect the fluid being treated in close proximity to the lamp. Baffles, however, can be expensive to construct inside of the reactor vessel, and can create regions of darkness along the baffle walls perpendicular to the radiation source.

U.S. Pat. No. 6,228,332 teaches a reactor vessel design that utilizes an annular baffle positioned at the inlet and a reflective concentric baffle positioned within the vessel for directing the fluid substantially parallel to the light source. A frustoconical baffle is also positioned at the outlet. This approach may effectively address hydraulic issues, but it has the disadvantage of being too expensive to construct for a typical individual-use type system. Additionally, the design does not lend itself to the production of small-scale systems because the region between the concentric baffle and vessel wall increases the size of the reactor without providing any effective dose, because the baffle is opaque.

A problem inherent to the use of flash-lamps for disinfection is that a flash-lamp needs to be driven hard to efficiently produce UV light. Thus, flash-lamps often suffer from short lifetimes. For example, a flash-lamp may require replacement every one to three months, while a comparable MP mercury UV lamp may require replacement once per year.

A miniaturized personal-use-type PUV disinfection system that is constructed inexpensively and operated intermittently at a point-of-use, such as a household faucet, at ½ gallon-per-minute (GPM) for 10 minutes every day can provide a household with an ample 5 gallons of disinfected water per day. A lamp that provides 1 to 3 months of continuous operation would conceivably last for 11 to 35 years under these conditions. Moreover, the efficiency of a typical mercury vapor lamp is significantly reduced as the lamp is scaled down. This is generally not the case with a flash-lamp.

For example, an industry leading 3-5 GPM water disinfection system utilizes a 27 watt low-pressure-high-output (LPHO) mercury vapor lamp that is 13.5″ long. It is known that a LPHO lamp can be about 35% efficient. To miniaturize such a system to ⅓ scale, the overall length of the lamp becomes 10 cm. Such commercially available specialty lamps have an efficiency of 0.25% or less and an input power of 1 W. A comparison of a PUV lamp to such a miniaturized mercury vapor lamp, assuming both are 5 mm in diameter, shows that a PUV lamp is many times more powerful. Based on the power density, photometric efficiency and UV efficiency numbers used previously, the PUV lamp can produce 214.9 WUV.
(π×0.5 cm×10 cm)×240 W/cm²×50%×11.4% UV=214.9 WUV

The PUV lamp makes 85,960 times more power (214.9 WUV/[1 W×0.25%]) than the miniaturized mercury vapor lamp of the same dimensions. This is based on the same numbers that illustrated the potentially inferior nature of PUV compared to large conventional MP mercury UV systems above. However, it is unnecessary to operate at such extremes and the cooling requirements can be relaxed. Additionally, the example illustrates that the power density exists to construct water disinfection devices with substantially smaller lamps and miniaturized reactor vessels that can provide an effective dose. Specifically, decreasing the vessel volume, and consequently the residence time, requires an increase in flux to achieve an equivalent dose.

Consequently, an ongoing need exists for a cost-effective, rugged, miniaturized, mercury-free system, and a corresponding method, for instantly disinfecting intermittently flowing fluids at the point-of-use.

### SUMMARY

Systems and processes for disinfecting fluids, such as water, use a mercury-free source of ultraviolet radiation. The mercury-free source of ultraviolet radiation is preferably a flash-lamp. The flash-lamp preferably has a lamp-resistance parameter (Kₒ) that is no greater than about 28 ohm-ampere^{1/2}, and more preferably between about 1.0 ohm-ampere^{1/2} and about 15 ohm-ampere^{1/2}. The systems and processes can be used, for example, to inactivate pathogens such as bacteria, spores, and viruses, and pyrogens such as endotoxin in the fluids. The systems and methods can irradiate the fluid with the mercury-free source of ultraviolet radiation in doses of, for example, about 1 mJ/cm² to about 300 mJ/cm².

### BRIEF DESCRIPTION OF THE DRAWINGS

FIG. 1 is a schematic overview of an embodiment of a system for disinfecting fluids;

FIG. 2 is a data table of theoretical radiation calculations for the system shown in FIG. 1;

FIG. 3 is a graphical representation of the plank joule distribution per unit wavelength over the UV spectrum for a single pulse of the system shown in FIG. 1;

FIG. 4 is the current wave shape of the optimized parameters and trigger method of the system shown in FIG. 1;

FIG. 5 is the current wave shape of the critical parameters in the ‘simmer’ mode of operation of the prior art;

FIG. 6 is the current wave shape of the critical parameters of with a series trigger method of the prior art;

FIG. 7 is a cross-sectional side view of a vessel of the system shown in FIG. 1;

FIG. 8A is an exploded view of the vessel shown in FIG. 7;

FIG. 8B depicts a portion of vessel shown in FIGS. 7 and 8A sectioned through the line “A-A” shown in FIG. 8A;

FIG. 9 is an illustration depicting top views of a top, mid, and bottom portion of an alternative embodiment of the vessel shown in FIGS. 7 and 8;

FIG. 10 is an illustration is an illustration depicting top views of a top and bottom portion of another alternative embodiment of the vessel shown in FIGS. 7-8B; and

FIG. 11 is an illustration of a dose accumulated by a pathogen traveling through the vessel shown in FIGS. 7-8B.

### DETAILED DESCRIPTION OF ILLUSTRATIVE EMBODIMENTS

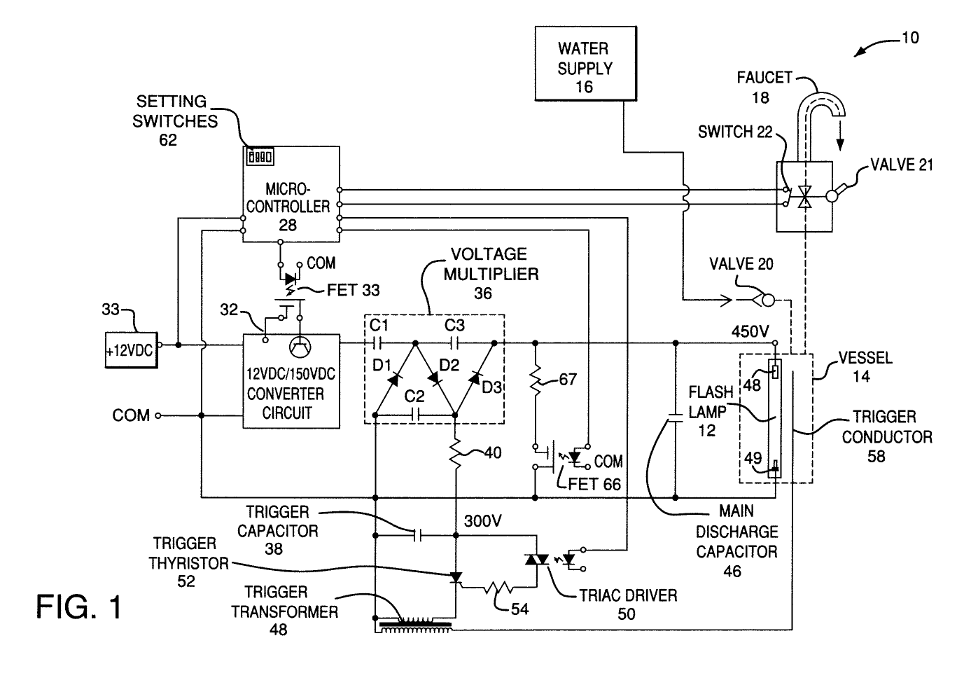

FIGS. 1, 7-8B, and 10 depict an embodiment of a mercury-free pulsed UV disinfection system 10, and various components thereof. The system 10 can be installed and operated at point of use intermittently-operated devices such as faucets in homes and offices without public water or hotels overseas or points-of-use in appliances, medical devices, pharmaceutical process equipment and gravity fed or pumped portable applications. The use of the system 10 is not limited, however, to these specific applications. Moreover, the flow rate of the process fluid through the system 10 as described herein is about 0.5 gallon per minute (GPM). This particular flow rate is specified for exemplary purposes only; the flow rate of the process fluid can be less than or greater than 0.5 GPM in alternative embodiments.

The system 10 includes a mercury-free flash-lamp 12 that functions as a source of pulsed-germicidal radiation. The use of a flash-lamp as the source of pulsed-germicidal radiation is disclosed for exemplary purposes only. Other types of pulsed-germicidal radiation sources, such as a surface-discharge or excimer-type lamp, can be used in lieu of a flash-lamp in alternative embodiments.

The electrodes of flash-lamps, in general, are rugged, and can be more durable than a conventional flashlight bulb. Moreover, flash lamps can be operated intermittently without significantly reducing the life thereof. The filament of a mercury vapor lamp, by contrast, is relatively fragile, and frequent power cycling of a mercury vapor lamp can significantly reduce filament life.

Flash lamps, in general, are substantially unaffected by operating temperature, and are instantaneously efficient. Also, the power of flash lamps can be adjusted. Conventional mercury vapor lamps, by contrast, generally require up to several minutes to reach full efficiency. Moreover, the efficiency of a mercury vapor lamp can be affected by operating temperature, and mercury vapor lamps generally have only one power.

The lamp 12 utilizes a luminous component such as xenon, krypton, or another noble gas to produce germicidal radiation. These types of materials do not present a substantial health risk to the user if the lamp 12 ruptures. Moreover, the lamp 12 does not need to be disposed of or shipped using specialized procedures for hazardous or toxic material, in contradistinction to mercury vapor lamps. Lamps that contain mercury, by contrast, present a substantial health hazard to the user upon rupturing, and usually must be shipped and disposed of using potentially burdensome procedures due to the toxic nature of the mercury.

Moreover, although large-scale mercury vapor lamps are generally more efficient than flash-lamps in generating germicidal radiation from input energy, the efficiency of mercury vapor lamps can decrease significantly as the dimensions of the lamp are reduced. Thus, it is believed that the system 10 can be constructed on a smaller scale than a system that uses a mercury vapor lamp, since a miniaturized lamp such as the lamp 12 can provide substantially greater power density and germicidal energy conversion efficiency than a mercury vapor lamp of the same dimensions. The lamp 12 can potentially produce tens of thousands of times more germicidal energy than the miniaturized mercury vapor lamp of about the same dimensions.

Exemplary operating parameters for the system 10 can be developed by initially considering the smallest flash-lamp that can be constructed from commercially available components and will produce wall-stabilized plasma. A wall stabilized operation is useful because the diameter of the arc during the peak discharge is fixed and estimated as the inner-diameter of the quartz envelope rather than varying in size when the voltage and capacitance are modified. Providing for an arc of known diameter allows for a more accurate estimation of Kₒ and other crucial factors for estimating the photometric efficiency and germicidal spectra such as the current density and plasma temperature.

The best photometric efficiency, it is believed, can be obtained by wall-stabilized flash-lamps of

$$10 \geq \frac{1}{d} \geq 5$$

l—Flash-Lamp Length

d=Flash-Lamp I.D.

Commercially-available flash-lamps for strobe and other applications have envelopes that are as small as 4 mm in inner diameter. Therefore, the minimum arc length for a 4 mm lamp that is wall stabilized is about 2 cm, where

$$\frac{l}{d} = 5.$$

The lamp 12 preferably has a fill pressure of about 450 torr. This value, it is believed, provides a relatively high luminous efficiency without making the lamp 12 problematic to ionize. With this, the dynamic resistance parameter of the lamp is calculated by:

$${K}_{o} ( Ω {A}^{1 / 2} ) = 1.27 ( \frac{20 mm}{4 mm} ) {( \frac{450}{450} )}^{1 / 5} = 6.350$$

Gas discharge lamps generally require an access point for gas filling. Such access points are commonly referred to as tip-offs. The anomalous surface of the tip-off occupies a significant portion of lamps with very small surface areas, such as the lamp 12, and impedes the UV radiation if placed between electrodes. For this reason, a tip-off 14 of the lamp 12 is preferably located behind the anode 48, or more preferably, behind the cathode 49 of the lamp 12.

The additional parameters of the system 10 are developed from the above value of Kₒ. The noted value for Kₒ represents the lowest theoretical impedance of all wall-stabilized flash-lamps of equivalent fill pressure that can be constructed from commercially-available 4 mm electrodes and envelope materials. However, the Kₒ of alternative embodiments can range from about 1 ΩA^{1/2} to about 28 ΩA^{1/2}, and is preferably less than about 15 ΩA^{1/2} as defined by the theoretical relationship above. It should also be noted that operating in a wall stabilized regime is not necessary, as the inventors have also achieved good photometric efficiency and substantial disinfection of water in alternative embodiments having an l/d of less than two.

The additional required parameters are as follows:

α—Damping Coefficient=0.76 (for critical damping with highest peak current)

t_{1/3}—Full Pulse Width (μs) at ⅓ height=28

E—Pulse Energy=6 J

The damping coefficient is an empirical constant used to calculate critical values of voltage, capacitance and inductance that will result in a current pulse shape that is not over-damped or will not oscillate. This helps to insure that the power is delivered to the lamp 12 with optimum efficiency, to produce the highest current density while reducing the stress on the lamp 12 and power supply components, thereby increasing operating life. The time constant is measured at the pulse width at ⅓ pulse height, and is chosen to be brief to minimize heat transfer from the plasma to the quartz envelope. Additionally, the time and energy values have been selected to produce values of voltage and capacitance that can be supplied by standard and miniaturized components as discussed below.

For any given values of t_{1/3} and E there is only one combination of capacitance (C), Inductance (L) and Voltage (V) values that will result in a critically damped current waveform with the highest amplitude (α=˜0.76) for a flash-lamp with a specific value of Kₒ. The critical capacitance, voltage and inductance are calculated as follows.

$$C \text{-} Capacitance ( μ F ) = {[ \frac{2 \times 6 J \times {.76}^{4} {( \frac{28 {E}^{- 6} sec}{3} )}^{2}}{6.350 Ω {A}^{1 / 2}} ]}^{1 / 3} \times \frac{1 {E}^{6} μ F}{1 F} = 60$$

$$L \text{-} Inductance ( μ H ) = \frac{{( \frac{28 μ sec}{3} )}^{2}}{60 μ F} = 1.4 \\ V \text{-} Voltage ( V ) = {( \frac{2 \times 6 J}{60 {E}^{- 6} H} )}^{1 / 2} = 450$$

The above parameters are the optimized operating parameters required for the voltage production and main discharge circuits of the system 10.

The system 10 comprises a reactor vessel 14 having the lamp 12 disposed therein. The fluid to be disinfected, e.g., water, is irradiated by the lamp 12 as the fluid flows through the vessel 14. The inlet of the vessel 14 is in fluid communication with a source of liquid such as a water supply 16. The outlet of the vessel 14 can be in fluid communication with a faucet 18 or other device that permits access to the disinfected water on a selective basis. The faucet 18 is positioned at or near the outlet of the vessel 14, so that the system 10 acts as a point-of-use disinfection system.

A true point-of-use system should serve as a barrier to viable pathogens. The system 10 can include a flow-operated valve 20 located in the flow path between the water supply 16 and the vessel 14. The valve 20 can be, for example, a check valve of a reed valve. The valve 20 can alternatively be located at the inlet or outlet of the vessel 14, or in the flow path between the vessel 14 and the faucet 18. The valve 20 can provide isolation to prevent viable pathogens from propagating downstream of the vessel 14 when the liquid is not flowing through the vessel 14. The valve 20 is selected so as to be self-actuated when the liquid is flowing through the vessel 14 during operation of the system 10.

Initiation of liquid flow through the vessel 14 and the faucet 18 is accomplished by a valve 21 located at the point of use, i.e., on the faucet 18. The valve 21 can be actuated between its open and closed positions on a manual basis by the user.

The system 10 also includes a switch 22. The system 10 can also include a switch 22, and a microcontroller 28. The switch 22 sends a signal to the microcontroller 28 as the liquid flow commences due to actuation of the valve 21. This can be accomplished by configuring the switch 22 so that a contact within the switch closes as the flow commences, thereby sending a logic-level high signal to the microcontroller 28. The switch 22 can be connected to and actuated by the valve 21. Alternatively, the switch 22 can be a flow or pressure switch. The use of this type of switch can potentially eliminate the need to modify the valve 21 or the faucet 18 to accommodate the switch 22. Alternatively, an analog signal from a flow meter can be utilized in applications where the liquid flow rate is variable.

A dump circuit of the system 10 is isolated immediately following the signal from the switch 22. The dump circuit 10 can include an opto-isolated depletion-mode field effect transistor 66, and a resistor 67 electrically connected to the field effect transistor 66. The isolating function can be accomplished by sending a logic-level high signal from the microcontroller 28 to the field effect transistor 66 or, alternatively, to a normally-closed relay. A logic-level high signal to a depletion-mode field effect transistor, in conjunction with a logic-level high signal to activate a DC inverter 32 of the system 10, as described below, is believed to provide fail-safe operation. Fail-safe operation is achieved because the energy storage capacitors will discharge in the event of power loss using low energy semiconductor devices, thereby avoiding the degradation that can occur in a mechanical switch due to arcing; and operation of the microcontroller 28 is required to produce and store voltage.

Following a brief delay, such as 1 ms, to allow the dump circuit to isolate, the microcontroller 28 activates the DC inverter 32, preferably by sending a logic-level high signal to an opto-isolated field-effect-transistor 33 on the gate of the high frequency oscillator of the DC inverter 32. This activation method eliminates the requirement of an additional substantial semiconductor or mechanical switch in the main discharge circuit.

The DC inverter 32 converts a relatively low voltage input from a power source 33 to a higher-voltage output. In the system 10, the low-voltage input is about 12V and the higher-voltage output is about 150 V. These particular voltage levels are specified for exemplary purposes only, and can vary by application.

The DC inverter 32 is electrically connected to a voltage multiplier 36. The 150V-output generated by the DC inverter 32 is doubled by a second stage of the voltage multiplier 36. The voltage multiplier 36 is electrically connected to a trigger capacitor 38, a resistor 40, and a main discharge capacitor 46. The trigger capacitor 38 is charged to about 300V through the resistor 40. The main discharge capacitor 46 is charged to about 450V from the third stage of the voltage multiplier 36.

The use of a voltage multiplier such as the voltage multiplier 36 is shown for simplicity, to illustrate a source of two potentials required by the main discharge capacitor 48 and the trigger capacitor 38. The trigger capacitor 38 is required when using a standard external trigger transformer 48 as shown in FIG. 1. The two potentials can alternatively be provided by separate windings on the DC inverter 32, by a voltage divider from main discharge capacitor 46, or by tapping the bottom potential of two main discharge capacitors in series. Additionally, the voltage of the trigger capacitor 38 can be adjusted to the same value of the main discharge capacitor 46 by appropriately modifying the trigger transformer 48.

The above-noted voltages can be generated by using a higher turns-ratio on the transformer of the DC inverter 32 to produce a higher potential than will be utilized. A voltage comparator, not shown for simplicity of illustration, can be used to provide a signal to the microcontroller 28 that the desired voltage has been reached. The signal can be generated, for example, by turning on an opto-isolated field effect transistor. The microcontroller 28, in turn, stops the DC inverter 32 by, for example, sending a logic-level low signal to the opto-isolated field-effect-transistor on the gate of the high frequency oscillator of the DC inverter 32.

The potential of the main discharge capacitor 46 is connected to an anode 48 and a cathode 49 of the lamp 12. The potential of the main discharge capacitor 46 is held off by the un-ionized lamp 12.

After isolating the dump circuit and actuating the DC inverter 32, the microcontroller 28 times-out for a period of milliseconds to allow charging of the trigger capacitor 38 and the main discharge capacitor 46. The first pulse is delivered to the fluid in the vessel 14 within milliseconds of actuation of the valve 21 to its open position, as sensed by the switch 22. The first pulse is initiated by sending a logic-level high signal from the microcontroller 28 to an opto-isolated random-phase TRIAC driver 50. The TRIAC driver 50 connects the potential of trigger capacitor 38 to the gate of a trigger thyristor 52 through a resistor 54, thereby directing the current from trigger capacitor 38 through the primary winding of trigger transformer 52. This function can alternatively be performed with a field effect transistor.

The secondary winding of the trigger transformer 48 is of high turns ratio to the primary winding. The secondary winding produces a micro-second pulse of several thousand volts in a trigger conductor 58 which is located in proximity to the lamp 12. The high-voltage pulse in proximity to the lamp 12 causes the gas inside the lamp 12 to ionize so that the gas conducts current from the main discharge capacitor 46 though the lamp 12.

Continuous radiation is delivered to the liquid flowing through the vessel 14 during the period in which the valve 21 is in the open position by pulsing the lamp 12 at a user-selected frequency. Pulsing of the lamp 12 can be achieved by setting switches 62, or by a rheostat monitored by the microcontroller 28. The user-selected frequency modulates the time period that the microcontroller 28 waits between initiating the pulses as described above, to deliver the desired germicidal power. The system 10 is thus able to provide several different disinfection doses, such as a low dose for home use and a high dose for travel, and can be used throughout a range of flow rates. Consequently, one system such as the system 10 can be used in different and varies applications such as home faucet systems, appliances, and portable applications.

The power-supplying circuitry of the system 10, generally, must extinguish the lamp 12 due to the relatively low resistance of the lamp 12 resulting from the relatively low Kₒ value of the lamp 12. In particular, the relatively low resistance would allow constant ionization of the lamp with little voltage and current. Additionally, the relatively low resistance of the lamp 12 would short-circuit and damage the power supply. In the case of the system 10, which has a pulse width of less than 100 μs, a delay of 1 ms before and after the pulse is sufficient to extinguish the lamp 12. This delay can be achieved by timing-out the gate of the field-effect-transistor which provides the high-frequency oscillation in the DC inverter 32 from the microcontroller 28, preferably through an opto-isolator. Timing-out for a linear supply can be accomplished by timing out at a zero-crossing with a TRIAC, preferably with an opto-isolated zero-crossing TRIAC driver. This configuration can eliminate the associated power losses and expense of using a separate substantial transistor between the capacitor charging circuit and the main discharge capacitor that must handle the average power.

Following the deactivation of the valve 21 and the subsequent cessation liquid of flow, the microcontroller 28 is programmed to cause the delivery of at least one pulse, and more preferably, a plurality of pulses to the fluid residing in the vessel 14 after the point-of-use signal from the switch 22 indicates that the flow has ceased. The purpose of the post-flow pulse or pulses is to disinfect the fluid that will be dispensed upon the subsequent activation of the system 10, and to disinfect the vessel to prevent pathogenic activity during the period when the system 10 is inactive.

The microcontroller 28 stops the DC inverter 32 following the final pulses. This can be achieved by sending a logic-level low signal to the opto-isolated field-effect-transistor on the gate of the high frequency oscillator of the DC inverter 32. The microcontroller 28 closes the dump circuit after a delay of, for example, about 1 ms. This can be accomplished by sending a logic-level low signal from the microcontroller 28 to the opto-isolated depletion-mode field effect transistor 66 of the dump circuit.

Operation of the system 10 can be controlled by means other than the microcontroller 28 in alternative embodiments. For example, the pulses can be initiated by a SIDAC such that the time constant of the pulse rate is adjusted by an RC circuit comprising the trigger capacitor and a potentiometer, or by one or more timing semiconductor devices. The use of a microcontroller such as the microcontroller 28 is preferred, however, as the microcontroller 28 can be used to facilitate additional functions for the system 10.

With the exception of the microcontroller 28, the system 10 does not consume power, such as leakage current from the capacitors, when the system 10 is not in use because the DC inverter 32 is off during periods of non-use. Moreover, the microcontroller 28 consumes only a fraction of a watt, and therefore does not consume a substantial amount of power. Alternatively, the power for the microcontroller 28 can be directed through the switch 22 so that the microcontroller 28 is dormant during periods of non-use of the system 10. The overall power consumption of the system 10 is thus believed to be substantially lower than that of disinfection system of comparable capability that operates on a continuous basis.

For example, the daily electricity consumption of the system 10, when operated intermittently in point-of-use applications, is believed to be less than 1/100th that of a conventional mercury vapor lamp of comparable capability. A mercury-vapor-based fluid disinfection system requires up to several minutes to reach full efficiency. In most applications, it is desirable that a user have instant access to the supply of disinfected water, at all times. The mercury based system therefore needs to be operated continuously, and thus will consume power continuously for 1,440 minutes per day. The system 10, by contrast, can provide instant access to disinfected water without being operated continuously. For example, the system 10 can disinfect ½ GPM at a faucet, and in supplying 5 gallons of water per day to a household, would consume power for only ten minutes per day (5 GPD/0.5 GPM), or for a time period that is 144 times less (1,440/10) than a continuously-operated system.

The lamp 12 and the power-supplying components of the system 10 can be constructed from relatively inexpensive and commercially available components, without a need for specialized parts such as those used in the laser industries. For example, the 450-volt operating voltage of the power supply components is within the operating range of many inexpensive and commercially available power supply components such as transistors, opto-isolators, and diodes. Moreover, this voltage can easily be supplied directly from a transformer or from a switched mode circuit with a high-frequency oscillator, miniaturized transformer, or inductor, or in combination with voltage multipliers. For example, many flash circuits utilized for portable digital cameras operate at this voltage. Additionally, many 450 volt metalized polypropylene capacitors are commercially available for motor start and other applications, and a small-scale capacitor constructed of 3 mil film can hold off 600V.

The radiation source of a flash-lamp is exceptionally hot plasma having a temperature of many thousands of degrees Kelvin, and created in the brief period of the main capacitor discharge. Under these conditions, plasma can reach a constant emissivity. The surface area of the plasma column can therefore be modeled as a blackbody with a constant emissivity of less than unity, otherwise known as a greybody. The germicidal efficiency of the lamp 12 can be derived from the parameters generated thus far by utilizing the method presented as follows.

The total flux emitted by a blackbody is calculated by the Stefan-Boltzmann relationship as follows:

$$\frac{P}{A} = σ {T}^{4}$$

σ=5.67E⁻¹² Wcm²K⁴

For plasma in thermodynamic equilibrium, the electron temperature (Tₑ) is equal to the radiance temperature. Since A=πdl and P=E/t and E=Vi and V=±Kₒ|i|^{1/2}, the electron temperature for a flash-lamp with a given plasma temperature efficiency (eₚ) can be calculated as follows:

$${T}_{e } = {( \frac{{e}_{p} \times i ( \underline{+} {K}_{o} {\vert i \vert}^{1 / 2} )}{σ π dlt} )}^{1 / 4}$$

It is generally known that flash-lamps can convert between about 50% to about 60% of input energy to radiation and that hot plasma can have an emissivity (\acute{ε}) of about 0.98. For the conservative case of a flash-lamp converting about 50% of input energy to radiation, the plasma temperature efficiency (eₚ) is about 0.51 (50%/0.98).

With the above equation, the dynamic plasma temperature of the lamp 12 can be calculated from the corresponding value of current (i) for each time interval (t) by integrating the equation below for the critical values of the preferred embodiment of the present invention of C=60 μF, L=1.4 μH, Vₒ=450, Kₒ=6.35 and a time interval of 1 μs.

$$L \frac{d I}{d t} \underline{+} {K}_{o} {\vert i \vert}^{1 / 2} + \frac{1}{C} {\int }_{0}^{t} I ⅆ τ = {V}_{o}$$

The calculated total flux emitted by the lamp 12 will equal about 50% of the input energy when the electron temperature and emissivity constant are plugged back in to the Stefan-Boltzman equation as below:
All Φ(W/cm²)=\acute{ε}×σ×Tₑ⁴

However, the plasma will also exhibit the same spectral radiance of a blackbody radiator according to Planck's law. With this relationship, the germicidal content (200-300 nm) in all flux for each time interval can be calculated as follows:

$$200 - 300 nm Φ ( W \text{/} {cm}^{2} ) = {ɛ}^{′} \times \% {T}_{uv} \times {\int }_{.200 \times {10}^{- 9} m}^{300 \times {10}^{- 9} m} [ \frac{2 π h {c}^{2}}{{λ}^{5}} ( \frac{1}{{ⅇ}^{h c \text{/} λ K {T}_{e}} - 1} ) ] ⅆ λ \times \frac{1 {m}^{2}}{1 \times {10}^{4} {cm}^{2}}$$

λ=Wavelength in meters

c=2.998×10⁸ ms (Speed of Light)

k=1.381×10⁻²³ J/K (Boltzmann's Constant)

h=6.626×10⁻³⁴ Js (Plank's Constant)

e=2.718 (Base of Natural Logs)

σ=5.67×10⁻⁸ Wm²K⁴

% Tᵤᵥ=95% (Transmittance of UV through quartz relative to all Φ)

The energy in joules (J) is calculated by multiplying the calculated watt values of the flux by the plasma column surface area and the time interval, as follows:
J(200-300 nm)=[200-300 nm Φ(W/cm²)]×t×πdl
J(All Φ)=[All Φ(W/cm²)]×t×πdl

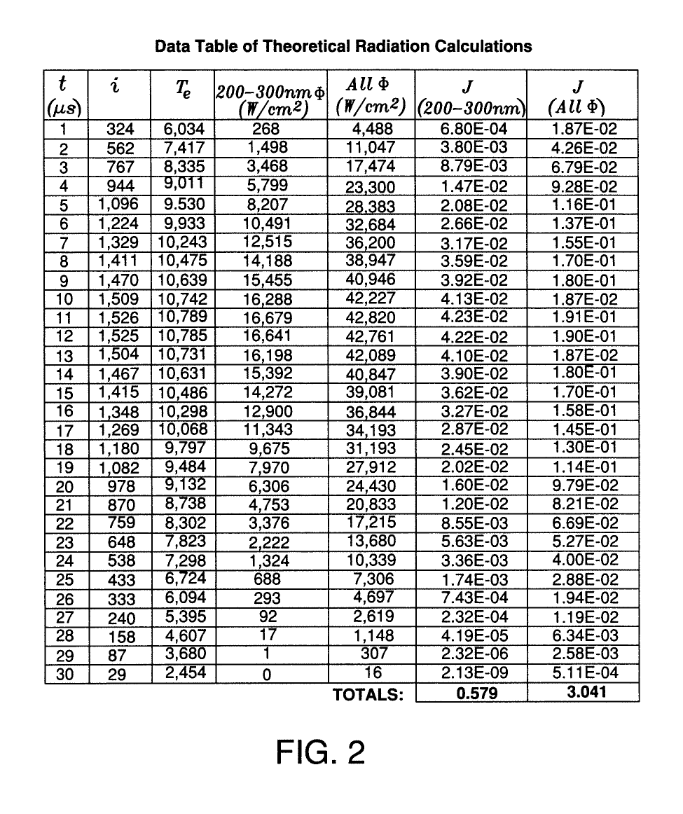

The data generated from the equations for the preferred embodiment of the present invention is summarized in the data table of FIG. 2 and the joule values are totaled.

The photometric efficiency is as anticipated at 50%, thus validating the calculations.

$$Input E ( J ) = \frac{60 {E}^{- 6} \times {450}^{2}}{2} = 6.075 J$$

$$\frac{3.041 J}{6.075 J} = 50 \%$$

The germicidal efficiency of the lamp and parameters of the system 10 is calculated at 9.5% and we have derived an absolute value of UV joules per pulse of 0.579 J.

$$\frac{.579 J}{6.075 J} = 9.5 \%$$

Because this radiation model is dynamic in nature, it produces results of spectral flux density per unit wavelength that do not correspond to spectra of blackbodies with a static temperature. A static temperature would only be applicable to continuous or square wave sources. Rather, the spectra for each time interval are added together.

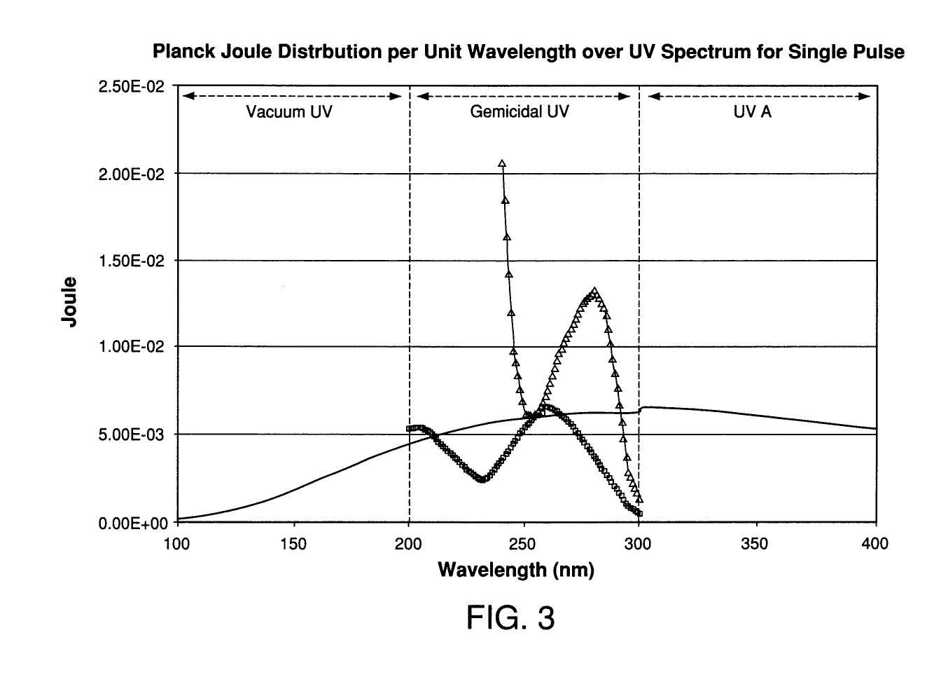

The Plank joule distribution per unit wavelength over the U spectrum, for a single pulse, is illustrated in FIG. 3. It is determined using the same blackbody calculations as above but in 1 nm increments. The joule distribution per pulse (solid line) is weighted by the absorption curve of DNA relative to 254 nm, and the result is represented by the symbols “□.” This is important because the majority of conventional data on disinfection doses and equivalent log inactivation rates are based on low-pressure mercury vapor lamps radiating exclusively at 254 nm, which matches very closely to the peak absorption of DNA at 260 nm. Therefore, the broad band germicidal radiation should be discounted by a germicidal weighting factor, and is determined by taking the ratio of the sum of the theoretical fluence values of the wavelengths between 200-300 nm before and after weighting by the absorption of DNA relative to 254 nm. With this method, the germicidal weighting factor is determined to be about 70%, and the total UV efficiency is adjusted as follows.
9.5%×70%=6.65% Theoretical Total UV Efficiency Relative to 254 nm

Moreover, although the theoretical PUV germicidal efficiency (represented by the symbol “□” in FIG. 3) is lower than that of a low-pressure mercury vapor lamp, the broad-band radiation of lamp 12 is believed to be more effective in inactivating a broader range of pathogens than mercury vapor lamps, particularly the LP or LPHO varieties. It is believed that the higher effectiveness of the lamp 12 is due to the emission powerful pulses of broad-band radiation with a continuum across the germicidal region of the electromagnetic spectrum from about 175 to about 320 nanometers (nm). Conventional mercury vapor lamps, by contrast, emit line radiation characteristic of mercury atoms in relatively cool plasma excited by an electrical charge. The primary mechanism for the inactivation of microorganisms is through the damage to cellular DNA and RNA resulting from the photochemical reactions initiated by the absorption of UV light at wavelengths of about 175 to about 320 nm. Bonds formed by adjacent pyrimidines in DNA and RNA are the principal forms of photo-damage caused by UV radiation. Although the absorption curve of DNA, with a peak at 260 nm, is generally weighed in determining the effectiveness of UV radiation wavelengths, pyrimidines thymine cytosine and uracil, the nucleotides involved in the formation of dimers and photoproducts, each have individual absorption curves. It is likely that variations in the composition of nucleotides in DNA and structures of microorganisms contribute to the significant disparity in the susceptibility of different strains of bacteria and viruses to mercury-based UV lamps that can only emit radiation at specific wavelengths. Because all chemical bonds of the organic molecules composing a microorganism require specific energies to trigger a photochemical reaction, a strong continuum of photon wavelengths is required to initiate a broad range of photochemical reactions.

Also, the broad-band radiation and high power density of flash lamps such as the lamp 12 are believed to be more effective on turbid fluid than conventional mercury vapor lamps. Although compounds in the fluid may absorb specific wavelengths, the broad-band radiation emitted by the lamp 12 ensures that other germicidal wavelengths will be transmitted to the pathogens. For example, a compound that absorbs strongly at 254 nm would incapacitate a conventional LP mercury disinfection system, but not the broad-band system 10. Additionally, a miniaturized system with high power density, such as the system 10, provides an equivalent dose to a larger scale mercury based system. Therefore, the path that the radiation travels is shorter than the paths traveled in larger-scale conventional systems. Consequently, the radiation should attenuate less in the system 10 than in conventional mercury based systems, in the presence of high-turbidity fluids.

Referring again to the theoretical spectra, the effect on proteins relative to 254 nm, represented by the symbol “⋄” in FIG. 3, is expected to be much more substantial. It is likely for this reason that photochemical reactions resulting from broad-band radiation may include denaturation of critical proteins such as those composing DNA repair enzymes and the metabolic functions of the organism. For example, it is known that polychromatic radiation has shown a suppressive effect on bacteria photo-reactivation. This may be caused by the denaturation of proteins composing the photolyase enzyme itself, or by broader forms of photo-damage to DNA that cannot be photo-repaired, such as cross-links or strand-brakes. Proteins exhibit a UV absorption curve from 175-320 nm with a weak absorption at 254 nm, the peak output of low-pressure (LP) mercury vapor lamps. It is most likely for this reason that LP mercury vapor lamps show little suppressive effect on bacteria photo-repair. This is significant because UV treatment does not provide residual disinfection as chemical treatment does.

Analogous to this example, the absorption curve of endotoxin illustrates that the theoretical spectra of the lamp 12 should strongly act on endotoxin in comparison with the radiation from a medium pressure mercury vapor lamp. Moreover, an LP mercury vapor lamp should have no effect at all. These proteins, which compose the cell wall of gram-negative bacteria, are as problematic as the pathogens themselves. In particular, endotoxins can cause septic shock and death, and are notoriously difficult to eliminate from fluids. Their small size makes them difficult to filter effectively, they can be transported through distillation and boiling endotoxin will not destroy them. The instantaneous elimination of endotoxin from fluids by the system 10 is beneficial to pharmaceutical and medical applications, such as water-for-injection and individual-use dialysis machines.

Referring to the data in FIG. 2, the peak current value is 1,525 A. This corresponds to a peak current density, an important factor in UV efficiency, of over 12,000 A/cm²:

$$\frac{1 , 525}{{π ( \frac{.4}{2} )}^{2}} = 12 \text{,} 128 A \text{/} {cm}^{2}$$

The system 10 can theoretically exceed the current density of large-scale PUV systems that typically operate at 6,000-10,000 A/cm². Consequently, it has been demonstrated that the system 10 can clearly equal or exceed the UV efficiency of such conventional systems without the expense of specialized components. Although a current density in excess of 12,000 A/cm² is exceptionally high, the theoretical parameters allow for inevitable circuit losses such as the ESR of the main discharge capacitor.

By integrating the following equation and plotting the current wave shape of the pulse as described previously, it is also possible to illustrate the characteristics of the trigger method of the system 10:

$$L \frac{d I}{d t} \underline{+} {K}_{o} {\vert I \vert}^{1 / 2} + \frac{1}{C} {\int }_{0}^{t} I ⅆ τ = {V}_{o}$$

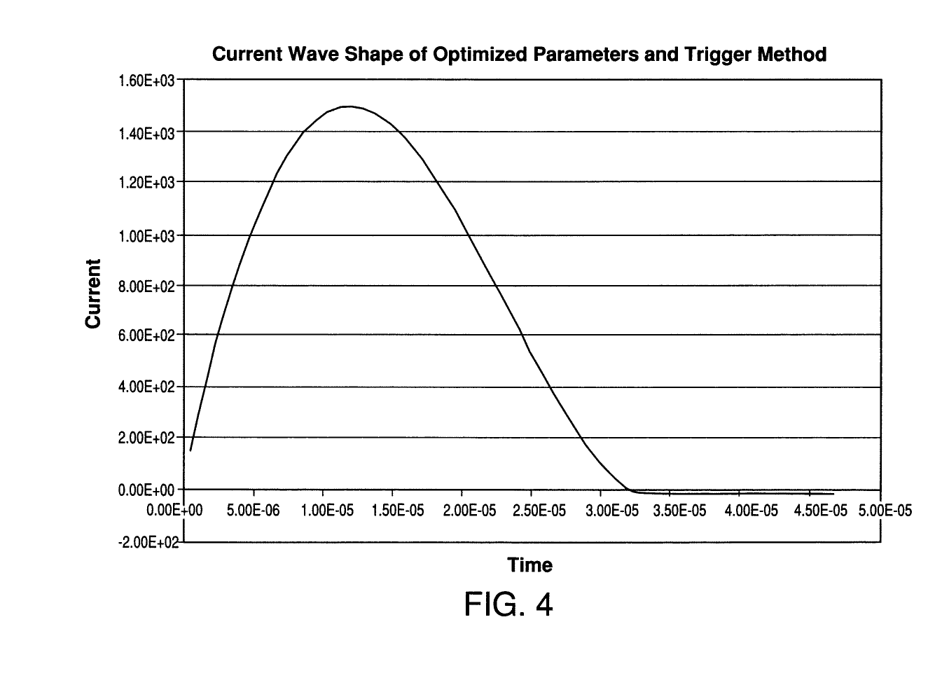

FIG. 4 is the current wave shape using the critical values of C=60 μF, L=1.4 μH and Vₒ=450 for Kₒ=6.35. The pulse duration at t_{1/3} is anticipated to be about 28 μs. The attack of the current wave is damped, which reduces the stress on the lamp 12, and is symmetrical with the decay. Additionally, a peak value of about 1,500 amps is achieved and the current returns to zero without oscillation. In this way, substantially all of the energy of the main discharge capacitor 46 has been delivered to the lamp 12 in the most efficient manner, and will produce the highest current density without overstressing the lamp 12. This will result in the highest plasma temperatures possible and, consequently, the most efficient UV production with the longest corresponding lifetime for the lamp 12.

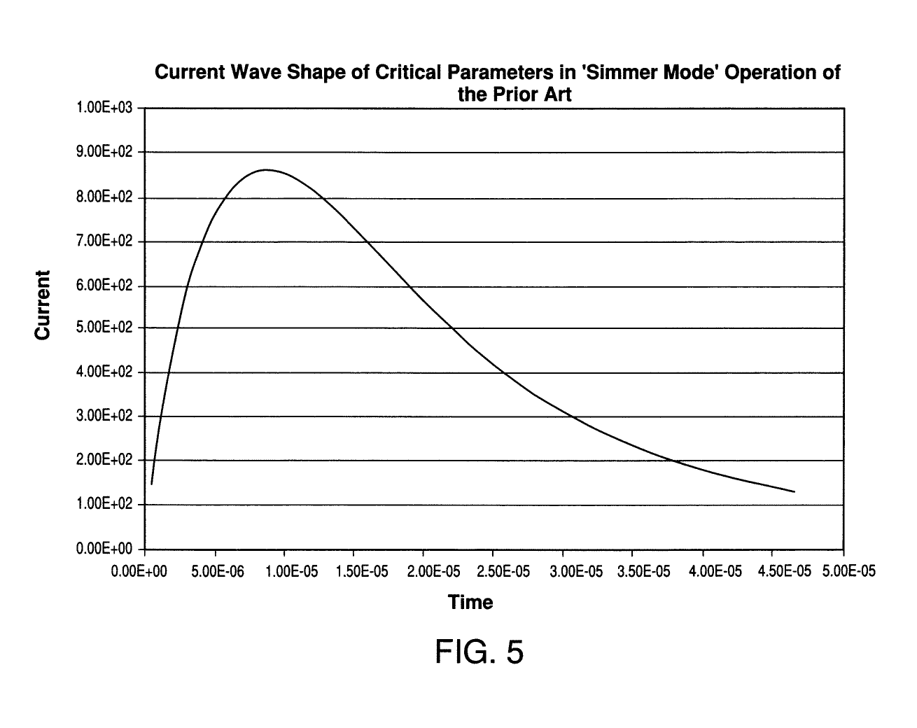

FIG. 5 represents the current wave shape using the same critical values as listed in FIG. 1, but introduces a term RI(t) into the immediately preceding equation. This term represents a static resistance of 0.2 ohms from a substantial semiconductor switch that must carry the peak discharge current as required in a “simmer” circuit as used in prior art PUV systems. Because the Kₒ value of the lamp 12 of the system 10 is relatively low, even a small resistance on the order of a fraction of an ohm in the discharge circuit will prevent the formation of a critical pulse current wave shape. In this case, the peak current is attenuated from about 1,500 amps to about 850 amps, and the duration of the pulse is elongated. Consequently, the stored energy from the main discharge capacitor 46 is not delivered to the lamp 12 in a manner conducive of the efficient production of UV, which requires high current density without over-stressing the lamp 12. Additionally, over half of the input energy has been lost to the switch, and is dissipated as heat as illustrated by the following equation:

$$\frac{.2 Ω}{{( \frac{1.5 μ H}{60 μ F} )}^{1 / 2} + .2 Ω} = 56 \%$$

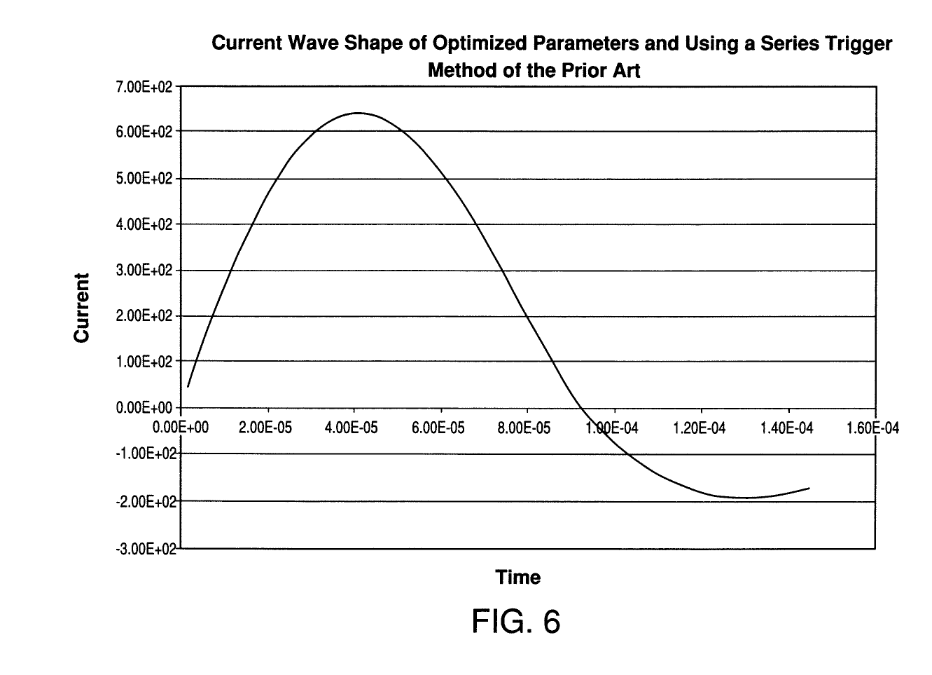

FIG. 6 depicts the current waveform of the critical parameters as in FIG. 1, except that the inductance is replaced with 14 μH to represent a minimum value that can be achieved in the secondary winding of a series trigger transformer of a conventional PUV lamp, where the turns ratio of the secondary winding to the primary winding on an iron core is relatively high. In this case, the peak current is attenuated from about 1,500 amps to about 640 amps. Additionally, the current will oscillate, damaging the polarized lamp 12, stressing the main discharge capacitor 46, and insuring that the energy will again be delivered to the lamp 12 in an ineffective manner.

Thus, triggering a flash-lamp with a low Kₒ value, such as the lamp 12 of the system 10, can be accomplished beneficially using the trigger method disclosed herein. The trigger should be external to the discharge circuit of the lamp 12, because any internal trigger mechanism can add unwanted resistance and/or excess inductance, and can negatively impact the UV efficiency of the lamp 12.

Initiating each main discharge with a high voltage pulse from an external trigger circuit can provide a relatively high germicidal efficiency, because the power required to maintain a simmer is eliminated. It is believed that this characteristic can increase the total germicidal efficiency of the system 10 by a minimum of about 10% in relation to conventional PUV lamps, and can extend the service interval for the system 10 by tens of times by allowing for operation at a lower-frequency pulse.

Additionally, the configuration of the system 10 does not need an isolation switch, such as a thyristor, MOSFET or IGBT, between the main discharge capacitor 46 and the lamp 12, in contradistinction to systems that relay on a simmer method for triggering a flash-lamp. A switch of this type can represent a significant expense, as the switch needs to be capable of holding off several thousand volts from the main discharge capacitor 46, and delivering several thousand amps during the peak of the discharge. Also, it is believed that the elimination of said isolation switch method may increase the germicidal efficiency of the system 10 by an additional 50% in relation to disinfection systems that incorporate a simmer, because the lamp 12 has a resistance value that is equivalent to the low resistance specialized switches utilized in conventional simmer circuits.

Moreover the trigger transformer 48 of the system 10 is substantially smaller and less expensive than the series trigger transformer of conventional simmer circuits because the secondary winding of the transformer, which is of high turns ratio to the primary winding, does not carry the main discharge current.

The trigger method used in the system 10 can permit a critical value of inductance in the lamp discharge circuit to be achieved. Because the secondary winding of conventional series trigger transformers is composed of a high number of turns over an iron core for the purpose of transmitting flux from the primary to the secondary winding, a high value of inductance is created in the secondary winding. Generally, this saturated inductance of the secondary winding is used to critically dampen the pulse of prior art PUV lamp circuits. However, the inductance of such transformers can exceed the value required to critically dampen the pulse of the discharge circuit of the system 10, which utilizes a lamp 12 with a relatively low value of Ko, typically between about 1 Ohm Ampres1/2 and about 15 Ohm Ampres1/2.

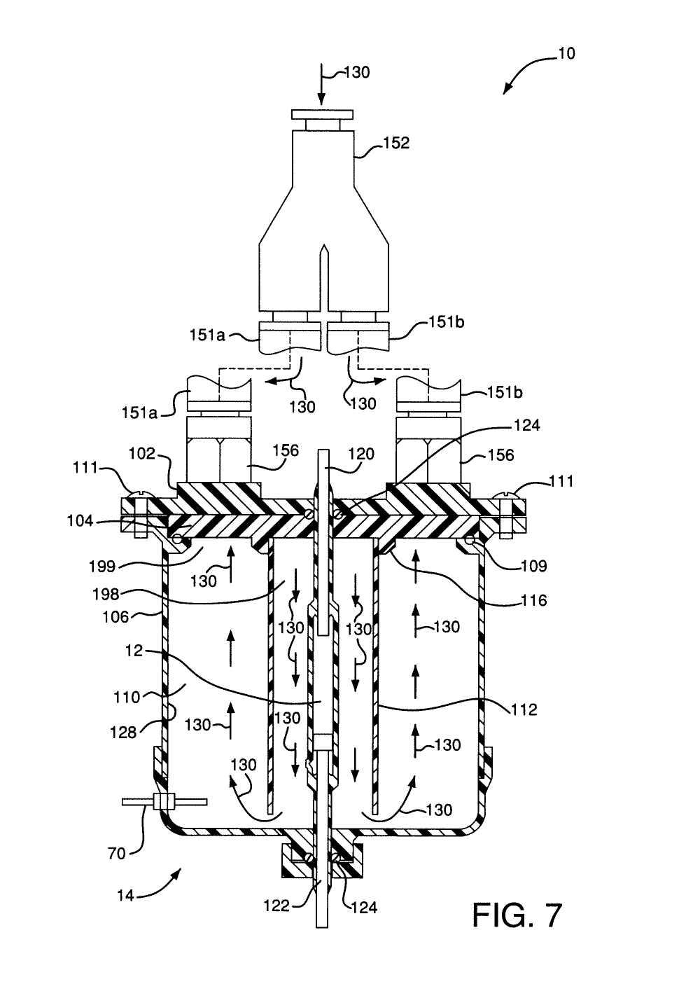

The trigger wire in the system 10 can be introduced into the vessel 14 by a connector 70, as shown in FIG. 7. The trigger wire can be encapsulated by, for example, a quartz capillary tube or silicon insulator. Alternatively, the trigger wire can be exposed directly to the process fluid, e.g., water, within the disinfection vessel. This is possible because the lamp 12 of the system 10 can consistently be triggered with little energy from the external trigger circuit, due to its relatively low Ko value. The relatively small surface area of the conductor required to trigger the lamp 12 inhibits immediate attenuation of the trigger voltage in even highly electrolytic fluids. Thus, the currents and voltages required to trigger the lamp 12 can be substantially less than the potential energy stored by the human body in a static discharge event. Therefore, the trigger method of the system 10 is safe for individual-use devices, and the conductor can be submersed directly in the process fluid, without insulation, in proximity to the lamp 12.

For example, the energy of the trigger capacitor of the system 10 is about 10 mJ as shown below. The arc from the trigger conductor can therefore be absorbed by one's finger without discomfort.

$$\frac{.22 {E}^{- 6} \times {300}^{2}}{2} = 9.9 m J$$

By comparison, the energy required to pose a risk to human health is 500 times greater at 5 J. In fact, the energy from human static discharge can vastly exceed this value because the capacitance of a human body is equivalent to the trigger capacitor of the system 10 at 200-300 pF, and static voltages can accumulate to 15,000 or 20,000V. Therefore, the energies from human static discharge can reach 22.5 J to 60 J, as shown by the following equations:

$$\frac{.200 {E}^{- 6} \times 15 \text{,} {000}^{2}}{2} = 22.5 J$$

$$\frac{.300 {E}^{- 6} \times 20 \text{,} {000}^{2}}{2} = 60 J$$

If the trigger wire is exposed directly to the process fluid, the electrolytic conduction of the high voltage pulse through the liquid to earth ground or common should be limited to levels corresponding to a few-thousand ohms of resistance and, more preferably, to at least 1 million ohms of resistance, for reliable operation of the system 10. This can be accomplished by utilizing non-conductive tubing, such as plastic, to introduce and withdraw the liquid from the vessel 14. The tubing should have a minimum bore area that can supply the desired flow rate, and a length adjusted to provide the required resistance. The conductivity range of the process fluid in a particular application should account for fluctuations of ion concentrations in the process fluid and differences in operating temperatures.

If the trigger wire is to be exposed directly to the fluid, the material from which the vessel 14 is constructed should be electrically non-conductive, or the vessel 14 should be isolated from earth ground or common.

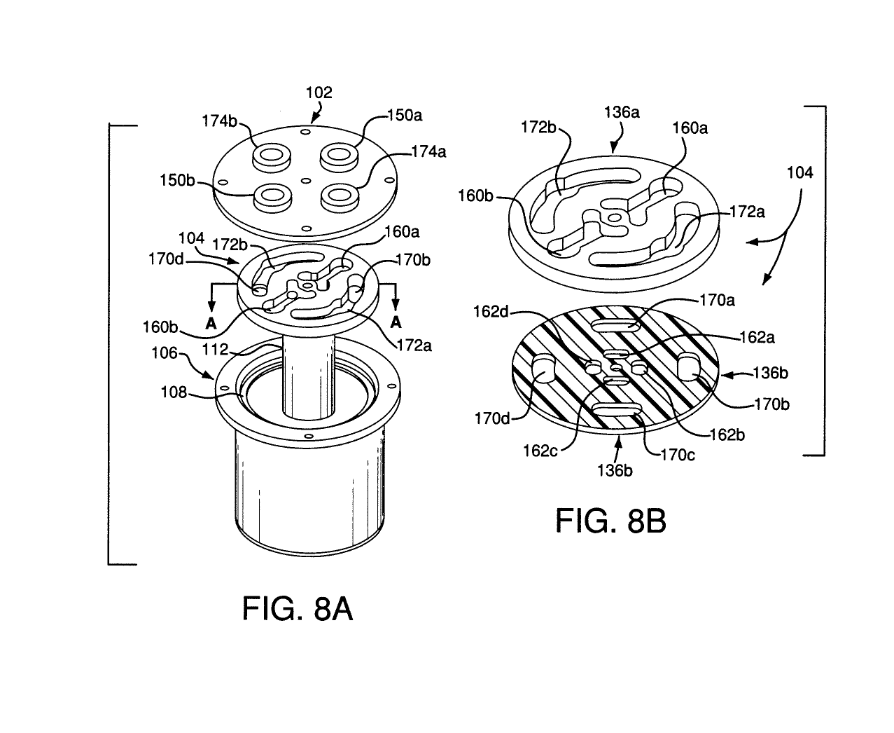

FIG. 7 is a cross-sectional side view of the vessel 14, and FIG. 8 is an exploded view of the vessel 14. The vessel 14 includes a top portion 102, a mid portion 104, and a bottom portion 106. The mid portion 104 is supported on an upwardly-facing, circumferentially-extending edge 108 of the bottom portion 106. The edge 108 is depicted in FIG. 8. The top portion 102 can be secured to the bottom portion 106 as depicted in FIG. 7, so that the mid portion 104 is trapped between the top portion 102 and the bottom portion 106. A gasket or other sealing means (not shown) can be disposed between the top portion 102 and the bottom portion 106.

The mid portion 104 and the bottom portion 106 define a volume 110 within the vessel 114. The surfaces of the mid portion 104 and the bottom portion 106 that define the volume 110 preferably are lined with a UV-resistant and reflective material such as polished stainless steel, chrome plated or painted plastic or, more preferably, with a non-conductive UV-reflective material such as GORE™ DRP®.

A transparent tube 112 is positioned substantially within the volume of the vessel 14. The tube 112 can be positioned so that the tube 112 and the bottom portion 106 are substantially concentric, as shown in FIG. 7. A first end 112a of the tube 112 is attached to a ring-shaped socket 116 of the mid portion 104, and the interface between the tube 112 and the socket 116 is sealed using a suitable means such as adhesive. The tube 112 can be formed from a material such as quartz, and preferably is formed from a deep-UV transparent quartz such as Suprasil™.

The lamp 12 is positioned substantially within the tube 112. The electrodes 113, 114 of the lamp 112 extend through the vessel 14 as depicted in FIG. 7. The interface between each electrode 120, 122 and the vessel 14 can be sealed using an O-ring seal 124 of other suitable means.

The lamp 12 and the inwardly-facing surface of the tube 112 define a first flow path for the process fluid. The process fluid, as discussed below, enters the first flow path from the mid portion 104 after being split into four separate and substantially equal flow streams.

The outwardly-facing surface of the tube 12 and the inwardly-facing circumferential surface 128 of the bottom portion 106 define a second flow path for the process fluid.

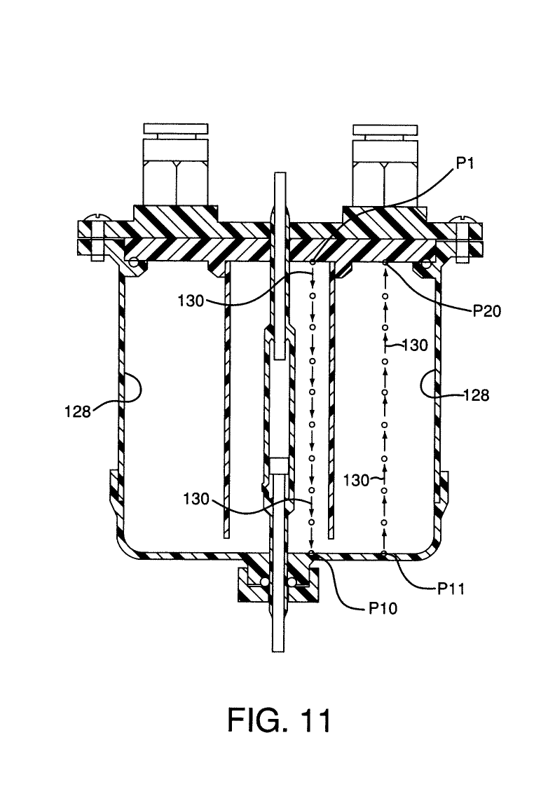

The general path of the process fluid through the volume 110 is denoted by the arrows 130 in FIGS. 7 and 11. It should be noted that the arrows 130 depict the general direction of flow of the process fluid and are not intended to denote all of the actual flow characteristics within the volume 110.

As denoted by the arrows in FIG. 7, the process fluid flows in a general downward direction along the first flow path defined by the lamp 12 and the tube 112. The process fluid is irradiated by the lamp 12 as it flows along the first flow path.

The process fluid exits the tube 112 upon reaching the second end 112b thereof. The process fluid subsequently turns and flows upwardly along the second flow path defined by the tube 12 and the surface 128 of the bottom portion 106. The process fluid is further irradiated as it flows along the second flow path.

The configuration of vessel 14 thus creates an overall flow path that is folded in upon itself to reduce short circuits without decreasing the effective treatment volume of the vessel 14, in contradistinction to opaque baffles used in prior art PUV systems, and in vessels having relatively low length-to-diameter ratios.

The process fluid exits the volume 110 as four separate streams upon reaching the end of the second flow path. The four streams, as discussed below, are subsequently combined into a single stream.

The vessel includes features that split the inlet flow into four separate and substantially equal streams, as discussed above. In particular, the top portion 102 includes a first inlet port 150a and a second inlet port 150b. The first and second inlet ports 150a, 150b are in fluid communication with a Y-type fitting 152 by way of respective first and second tubes 151a, 151b. The first and second tubes 151a, 151b are of substantially equal length. A T-type fitting can be used in lieu of the Y-type fitting 152.

Fittings 156 can be threaded, bonded, or otherwise connected to the first and second inlet ports 150a, 150b to facilitate connection of the tubing thereto. The Y-type fitting 152 and the fittings 156 are drawn to scale in relation to the vessel 14 in FIG. 7. The fittings 152, 156 can be conventional push-type fittings for ¼-inch tubing. Fittings of other sizes, e.g., 1/16-inch to 2-inch, can be used in the alternative. Moreover, other types of fittings, e.g., compression, face or O-ring seal, or barbed, can be used in the alternative.

The mid portion 104 can be molded as one piece from a suitable material such as plastic. FIG. 8B depicts the mid portion 104 sectioned into an upper half 136a and a lower half 136b.

The upper half 136a of the mid portion 104 has a first and a second inlet channel 160a, 160b formed therein. The first and second inlet channels 160a, 160b are substantially identical, and are disposed around the centerline of the mid portion 104 in a substantially symmetrical manner.

The first and second inlet channels 160a, 160b are shaped as depicted in FIGS. 8A and 8B. In particular, the first and second inlet channels 160a, 160b extend generally in a radial direction. A radially outermost portion of each of the first and second inlet channels 160a, 160b has a shape that substantially matches the shape of the first and second inlet ports 150a, 150b of the top portion 102. The outermost portions of the first and second inlet channels 106a, 106b overlap, i.e., align with, the respective first and second inlet portions 150a, 150b when the vessel 14 is assembled.

The process fluid thus enters the outermost portions of the first and second inlet channels 160a, 160b after passing through the respective first and second inlet ports 150a, 150b. The process fluid flows generally in a radially inward direction, i.e., toward the centerline of the vessel 14, in each of the first and second inlet channels 160a, 160b. The process fluid eventually reaches a radially innermost portion of each of the first and second inlet channels 160a, 160b is substantially arc-shaped, as shown in FIGS. 8A and 8B. The process fluid reaches the radially innermost portion of each of the first and second inlet channels 160a, 160b. The radially innermost portions of the first and second inlet channels 160a, 160b are substantially arc-shaped, as shown in FIGS. 8A and 8B.

The lower half 136b of the mid portion 104 has a first, second, third, and fourth inlet port 162a, 162b, 162c, 162d formed therein, as shown in FIG. 8B. The first, second, third, and fourth inlet ports 162a, 162b, 162c, 162d are substantially identical, are equally spaced from the neighboring first, second, third, or fourth inlet ports 162a, 162b, 162c, 162d, and are disposed around the centerline of the mid portion 104 in a substantially symmetrical manner.

The first and second inlet ports 162a, 162b substantially align with opposing ends of the radially innermost portions of the first inlet channel 160a. The third and fourth inlet ports 162c, 162d substantially align with opposing ends of the radially innermost portions of the second inlet channel 160b.

The first, second, third, and fourth inlet ports 162a, 162b, 162c, 162d are positioned above, and adjoin the volume defined by the tube 112 when the vessel 14 is assembled. The first, second, third, and fourth inlet ports 162a, 162b, 162c, 162d thus discharge into the upper region of the first flow path defined by the tube 112. This region is denoted by the reference character 198 in FIG. 7.

During operation of the system 10, the process fluid, e.g., water, is supplied to the Y-type fitting 152 from a source. The Y-type fitting 152 divides the flow into two streams of substantially equal flow rates. A first of the streams flows into the first inlet channel 160a of the mid portion 104 after passing through the first tube 151a and the first inlet port 150a of the top portion 102. The second stream flows into the second inlet channel 160b after passing through the second tube 151b and the second inlet port 150b.

The first stream of process fluid flows in a radially-inward direction through the first inlet channel 160a, and is splits into two secondary streams of substantially identical flow rates upon reaching the radially-innermost portion of the first inlet channel 160a. Each of the secondary streams flows downwardly, through an associated one of the first and second inlet ports 162a, 162b, and is discharged into the first flow path within the tube 12.

The second stream of process fluid is likewise split into two secondary streams of substantially identical flow rates upon reaching the radially-innermost portion of the second inlet channel 160b. Each of the secondary streams flow downwardly, through an associated one of the first and second inlet ports 162a, 162b, and is discharged into the first flow path within the tube 12.

The Y-type fitting 152 and the mid portion 104 thus divide the initial flow of process fluid so that four substantially identical and symmetrically-distributed flows of process fluid enter the region 198 of the volume 110. This feature helps to ensure that the process fluid is distributed around the lamp 12 in a substantially uniform manner as the process fluid flows through the vessel 14.

The lower half 136b of the mid portion 104 has a first, second, third, and fourth outlet port 170a, 170b, 170c, 170d formed therein. The first, second, third, and fourth outlet ports 170a, 170b, 170c, 170d are substantially identical, are equally spaced from the neighboring first, second, third, or fourth outlet ports 170a, 170b, 170c, 170d, and are disposed around the centerline of the mid portion 104 in a substantially symmetrical manner.

Each of the first, second, third, and fourth outlet ports 170a, 170b, 170c, 170d is located above, and adjoins a radially-outward portion of the volume 110. The first, second, third, and fourth outlet ports 170a, 170b, 170c, 170d therefore receive process fluid from the second flow path within the volume 110.

The upper half 136a has a first and a second outlet channel 172a, 172b formed therein. The first and second outlet channels 172a, 172b are substantially identical, and are disposed around the centerline of the mid portion 104 in a substantially symmetrical manner.

The first and second outlet channels 172a, 172b are substantially arc-shaped, as shown in FIGS. 8A and 8B. The first outlet channel 172a overlaps and adjoins the first and second outlet portions 170a, 170b. The second outlet channel 172b overlaps and adjoins the third and fourth outlet portions 170c, 170d.

The top portion 102 has a first and a second outlet port 174a, 174b formed therein. The first outlet port 174a overlaps and adjoins the first outlet channel 172a, and the second outlet port 174b overlaps and adjoins the second outlet channel 172b when the when the vessel 14 is assembled.

During operation of the system 10, the process fluid flowing generally upwardly along the upper region of the second flow path within the volume 110 enters the first, second, third, and fourth outlet ports 170a, 170b, 170c, 170d. The upper region of the second flow path is denoted by the reference character 199 in FIG. 7.

The flow streams entering the first and second outlet ports 170a, 170b subsequently enter the first outlet channel 172a, and flow toward each other within the first outlet channel 172a. The flow streams combine and exit the first flow channel as a single flow stream. The combined flow stream subsequently enters the first outlet port 174a of the top portion 102.

The flow streams entering the third and fourth outlet ports 170a, 170b subsequently enter the second outlet channel 172b, and flow toward each other within the second outlet channel. The flow streams combine and exit the first flow channel 172a as a single flow stream. The combined flow stream subsequently enters the second outlet port 174b of the top portion 102.

Fittings (not shown), such as the above-noted fittings 156 can be threaded, bonded, or otherwise connected to the first and second outlet ports 150a, 150b, to facilitate connection of tubing, such first and second tubes 151a, 151b, to the first and second outlet ports 150a, 150b. The tubing can carry the flow streams from the first and second outlet ports 150a, 150b to a fitting (not shown), such as the above-noted Y-type fitting 152, that combines the flow streams. The resulting stream disinfected process fluid can be directed to the faucet 18 by way of the valve 21.

The disinfected process fluid thus exits the region 199 of the volume 110 by way of four substantially identical exit points. The multiple flows are subsequently combined into a single stream by way of substantially identical flow paths. Manipulating the process fluid in this manner promotes uniformity in the flow of the process fluid through the volume 110.

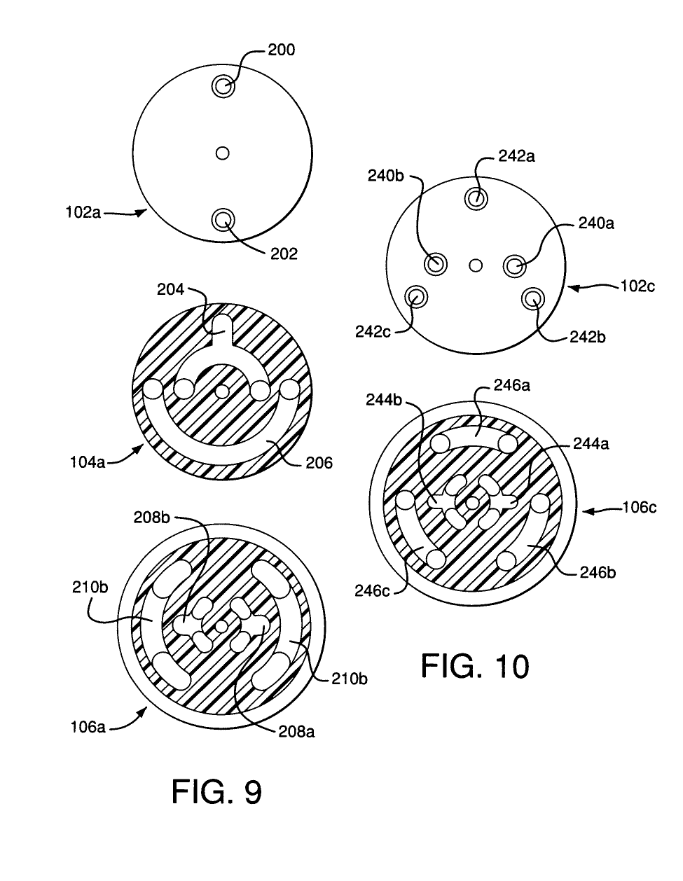

FIG. 9 depicts an alternative embodiment of the top portion 102, mid portions 104, and bottom portion 106 in the form of a top portion 102a, mid portion 104a, and bottom portion 106a. The top portion 102a, mid portion 104a, and bottom portion 106a divide the process fluid flowing into vessel 14 into four substantially equal streams, and combine the disinfected process fluid flowing out of the vessel 14 into one stream without the use of Y-type or T-type fittings.

The top portion 102a includes a single inlet port 200 and a single outlet port 202. The mid portion 104a includes a substantially Y-shaped inlet channel 204, and a substantially arc-shaped outlet channel 206. The inlet port 200 aligns with, and overlaps the inlet channel 204. The outlet port 202 aligns with, and overlaps the inlet channel 206.

The bottom portion 106a includes a first inlet channel 208a and a second inlet channel 208b. One end of the inlet channel 204 of the mid portion 104a aligns with, and overlaps first inlet channel 208a, and the other end of the inlet channel 204 aligns with, and overlaps second inlet channel 208a. The first and second inlet channels 208a, 208b each have two openings, at opposing ends thereof, that adjoin the volume 110.

The bottom portion 106a also includes a first outlet channel 210a and a second inlet channel 210b. One end of the outlet channel 206 of the mid portion 104a aligns with, and overlaps first inlet channel 210a, and the other end of the inlet channel 206 aligns with, and overlaps second outlet channel 210a when the vessel 14. The first and second outlet channels 208a, 208b each have two openings, at opposing ends thereof, that adjoin the volume 110.

The incoming process flow enters the inlet channel 204 of the mid portion 104a by way of the inlet port 200. The flow is divided into two streams by the inlet channel 204. Each stream enters an associated one of the first and second inlet channel 208a, 208b of the bottom portion 106a after reaching an associated end of the inlet channel 204. The first and second inlet channels 208a, 208b divide each stream into two additional streams which are discharged into the volume 110 by way of the openings in the first and second inlet channels 208a, 208b.

The disinfected process fluid flowing upward along the second flow path in the volume 110 enters first and second outlet channels 208a, 208b by way of the openings therein. The two separate flows entering each of the first and second outlet channels 208a, 208b by way of the two openings formed therein are combined. The resulting flow streams enter opposite ends of the outlet channel 206. The flow streams are combined in the outlet channel 206, and exit the vessel by way of the outlet port 202.

FIG. 10 depicts another alternative embodiment of the top portion 102, mid portion 104, and bottom portion 106 in the form of a top portion 102c and a bottom portion 106c. The top portion 102c includes a first inlet port 240a and a second inlet port 240b. The top portion 102c also includes a first outlet port 242a, a second outlet port 242b, and a third outlet port 242c.

The bottom portion 106c includes a first inlet channel 244a and a second inlet channel 244b. The first and second inlet ports 240a, 240b align with, and overlap the respective first and second inlet channels 244a, 244b. The first and second inlet channels 244a, 244b are substantially Y-shaped. Opposing ends of each of the first and second inlet channels 244a, 244b are open, and adjoin the volume 110. The open ends of the first and second inlet channels 244a, 244b are substantially identical, are equally spaced from the neighboring openings of the first or second inlet channels 244a, 244b, and are disposed around the centerline of the bottom portion 106c in a substantially symmetrical manner.

The bottom portion 106c also includes a first outlet channel 246a, a second outlet channel 246b, and a third outlet channel 246c. The first, second, and third outlet ports 242a, 242b, 242c overlap the respective first, second, and third outlet channels 246a, 246b, 246c. The first, second, and third outlet channels 246a, 246b, 246c are substantial arc-shaped. Opposing ends of each of the first, second, and third outlet channels 246a, 246b, 246c are open, and adjoin the volume 110. The open ends of the first, second, and third outlet channels 246a, 246b, 246c are substantially identical, are equally spaced from the neighboring openings of the first, second, and third outlet channels 246a, 246b, 246c, and are disposed around the centerline of the bottom portion 106c in a substantially symmetrical manner.

The inlet process flow can be split by a fitting (note shown), such as the Y-type fitting 152. The two resulting streams can be directed to the first and second inlet ports 240a, 240b by way of tubing (not shown), such as the first and second tubes 151a, 151b.

The substantially equal flow streams from the first and second inlet ports 120a, 120b enter the respective first and second inlet channels 244a, 244b. The flow in of the first and second inlet channels 244a, 244b is split into two substantially equal flows, each of which enters the volume 110 by flowing through the remainder of the first or second inlet channel 244a, 244b. The flow streams subsequently flow along the first flow path within the volume 110.

The disinfected process fluid flowing upward along the second flow path in the volume 110 enters the open ends of the first, second, and third outlet channels 246a, 246b, 246c by way of the openings therein. The two separate flows entering each of the first, second, and third outlet channels 246a, 246b, 246c by way of the two openings formed therein are combined. The resulting flow streams within the first, second, and third outlet channels 246a, 246b, 246c subsequently exit the vessel 14 by way of the respective first, second and third outlet ports 242a, 242b, 242c. The three flow streams can subsequently be combined by a suitable means such as two of the Y-type fittings 152.

The direction of flow is identified in the figures for illustrative purposes, and because the system 10 was operated in this manner during testing. The symmetry of the inlet and outlet configurations, however, can facilitate flow in directions opposite those noted in the figures. Moreover, the splitting and combining of the inlet and outlet flows can be achieved using more, or less that the number of channels and/or ports described above or, alternatively, exclusively though the use of flow splitters such as Y-type or T-Type fittings.

The system 10 does not create a substantial restriction when placed into the flow path of a fluid because the cross sectional area of the flow path through the inlet, vessel, and outlet channels can be greater than the fluid conduit connecting the system 10 to the source of the fluid and its point-of-use. Moreover, the division of the flow paths and the increase in the cross-sectional area of the channels relative to the fluid conduit decreases the velocity of the fluid in the region before the fluid communication ports of the vessel.

The disinfection efficiency of a reactor vessel is determined by integrating the germicidal flux throughout the vessel volume and multiplying by the residence time of the fluid passing through the vessel. Therefore, the highest disinfection efficiencies are believed to be achieved with reactor vessels approaching “plug flow” where complete mixing is assumed and residence time is determined as the volume divided by the flow rate. Large scale conventional reactors typically operate at about 60% to about 80% of plug flow.

$$t ( sec ) = \frac{Vol ( cc )}{cc \text{/} sec}$$

Since the volume of a cylinder of length (l) and diameter (d) equals π(d/2)² l and the velocity of the fluid moving through the cylinder parallel to its length is l/t, the fluid velocity of a constant flow rate is inversely proportional to the cross-sectional area of the cylinder:

$$\frac{l}{\frac{{π ( \frac{d}{2} )}^{2} l}{{cm}^{3} \text{/} sec}} or {( \frac{A}{{cm}^{3} \text{/} sec} )}^{- 1}$$

More specifically, it is desirable that the velocities in the fluid communication regions of the vessel [R-IN] and [R-OUT] of the present invention be as close to plug flow velocities as possible so that a minimum of time and distance is required to settle into a plug flow regime. This is because the vessel volumes of the embodiments of the present invention are small relative to conventional systems, but operate at comparable flow rates. Regions of high velocity relative to their surroundings can contribute to short circuits in the fluid or back mixing, and decrease the efficiency of the reactor vessel.

Thus, the total, i.e., combined, area of the first, second, third, and fourth inlet port 162a, 162b, 162c, 162d should be substantial in relation to the cross-sectional area of the region 198 within the volume 110, and the total area of the first, second, third, and fourth outlet ports 170a, 170b, 170c, 170d should be substantial in relation to the cross-sectional area of the region 199. The ratio of the velocity of the fluid traveling through the ports, to the plug flow velocity in the corresponding region of the vessel is directly proportional to the ratio of the areas respectively. However, this is only valid if the flow through a single port is substantially uniform and that flow relative to the other ports flowing in the same direction is also substantially uniform and, preferably, distributed in a substantially symmetrical manner by utilizing the previously described methods of dividing and re-directing the fluid.

The ratio of fluid communication velocity to plug flow velocity in the system 10 is preferably between about 1 and about 50, and more preferably, is between about 1 and about 10.

Because the present invention does not utilize auxiliary cooling for the radiation source, the flow path of the process fluid created between the radiation source lamp 12 and tube 112 from the first port and the second end of the tube 112 creates a high velocity jacket of cooling fluid over the lamp envelope and electrodes from the process fluid itself, and can eliminate the need for expensive auxiliary flash-lamp cooling systems that typically comprises a pump, a re-circulating loop, and intensive deionization and filtration components. The cooling efficiency of the system 10 is believed to be comparable to that of laser-lamp cooling systems.

For example, an industry standard laser cooling system is 10″ high×14″ long×10″ wide and costs over $2,000 because it utilizes intensive in-line filtration and deionization components. It provides about 1 kW of cooling capacity at 1.4 GPM. However, in the system 10, the flow rate of the process fluid is about 0.5 GPM. The adjusted cooling capacity can then be estimated as follows:

$$Adjusted Capacity ( W ) = \frac{( p \times .5 ) {C}_{p} Δ T}{( p \times 1.4 ) {C}_{p} Δ T}$$

p=Density

Cₚ=Heat Capacity of Water

ΔT=Outlet−Inlet Temp (K)
Assuming ΔT is roughly the same, the adjusted capacity of our device at 0.5 GPM is estimated as follows at about 35.7%, as follows:

$$\frac{.5 G P M}{1.4 G P M} = 35.7 \% of maximum capacity .$$

Water-cooled lamps can produce about 200 W/cm² of bore area. The internal area of the lamp envelope is 2.513 cm².

The maximum power range of the system 10 utilizing a standard quartz lamp envelope and flowing at about 0.5 GPM is estimated at about 180 W using an existing laser cooling system as a benchmark is:

$$2.513 {cm}^{2} \times \frac{200 W}{1 {cm}^{2}} = 502.6 W \times 35.7 \% = 179.4 W$$

This is a significant consideration because the system 10 needs to deliver a sufficient dose of germicidal radiation at the desired flow rate of the application because it does not rely on auxiliary cooling loops. Moreover, the cooling capacity of the system 10 is believed to scale with the flow rate. Disinfecting at a higher flow rate requires more lamp power and, consequently, a greater amount of cooling. The increased flow rate through the transparent tube 112 compensates for this. This is particularly well suited for embodiments which have adjustable power.

The majority of water cooled flash-lamps used in the optoelectronics industries have quartz envelopes that are shrunk down over the outer diameter of substantial lamp electrodes to provide efficient cooling to the electrodes, because quartz possesses good thermal conductivity, and as much as 40% of input energy can contribute to heating the electrodes. This method can be used in alternative embodiments requiring high power. Because embodiments of the present invention have a short gas discharge lamp arc-length to vessel-length ratio, the extended length of the substantial electrode encapsulated in quartz can provide the fluid-tight seal to the vessel or the lamp envelope can be expanded to the original and uniform size to produce the seal. The additional process of shrinking the quartz over the electrodes, however, can add significant cost to the manufacturing of the lamp.

Additionally, lower-power applications may only require air-cooled type electrodes that are less expensive to manufacture. In this case, a portion of the extended length of the electrode, created by the short arc-length to vessel length ratio, can be encapsulated in a material of high thermal conductivity and low electrical conductivity such as glass or quartz. With this method, the short arc-to-vessel length of the system 10 facilitates additional cooling for the electrodes by the process fluid, and provides thermal isolation between the electrodes and the exterior of the vessel. This configuration can thus help dissipate the heat generated by the electrodes, which can consume as much as 40% of the input energy to the system 10. In this case, the liquid-tight vessel seal can be provided by a ridged portion of the extended length of the electrode or a portion of flexible lead wire.

An estimate of the disinfection dose that can be delivered by system 10 can be determined to demonstrate the viability the system 10. The parameters required to perform such a proof-of-concept are as follows.

Summary of the Parameters Generated Thus Far:

| | |
|---|---|
| Lamp Length (l) - 2 cm | |
| Pulse Energy (E) - 6 J | % UV Efficiency (%_{UV})- 6.65 |

Additional Parameters Required for Dose Estimate:

| | |
|---|---|
| Vessel Length (l_{V}) - 5.87 cm | Vessel I.D.(d_{V}) - 6.10 cm |
| Lamp Outer Diameter (d_{L}) - .6 cm | Transparent Tube Dia. (d_{T})- 2 cm |
| Flow Rate (F_{R}) - .5 GPM | Pulse Frequency (Hz)- 5 Hz |
| % UV Transmission (%_{T})- 90/cm | % Plug Flow (%_{PF})- 60 |

FIG. 11 is an illustration of the dose accumulated by a pathogen traveling through the vessel 14, through the first path, midway between the lamp 12 and the inside of the tube 112, and the second path, midway between the tube 112 and the inwardly-facing circumferential surface 128 of the vessel 14. An approximation of the dose delivered can be obtained by averaging the dose accumulated by a pathogen at ten points along the first path, and at ten points along the second path. The various points along the path are denoted by the circles in FIG. 11. The circles between and including those labeled “P1” and “P10” are points located along the first flow path. The circles between and including those labeled “P11” and “P20” are points located along the second flow path.

Point source calculations are inaccurate at distances of less than five times the longest dimension of a radiation source. Therefore, the fluence within the region of the reactor vessel of system 10 at the ten points along each of the two paths can be determined by the following equation known as the LSI model which treats the lamp as an infinite number of point sources and approximates the fluence rate at any point of normal distance R from the lamp and a longitudinal distance H from the center of a lamp of arc length l.

$$I = \frac{P}{4 π lR} [ arctan ( \frac{l / 2 + H}{R} ) + arctan ( \frac{l / 2 - H}{R} ) ]$$

$$P = E \times Hz$$

The dimension R is constant for each of the two paths and is determined as follows.

$${R}_{1} ( Path 1 ) = \frac{{d}_{T} - {d}_{L}}{4} + \frac{{d}_{L}}{2}$$

$${R}_{2} ( Path 2 ) = \frac{{d}_{V} - {d}_{T}}{4} + \frac{{d}_{T}}{2}$$

The H dimensions will be the same for the two paths, and the first five points of each path are symmetrical with the last five points. The required H dimensions are determined as flows.

H₁=l_{V}/2

H₂=H₁−(l_{V}/9)

H₃=H₁−(2×l_{V}/9)

H₄=H₁−(3×l_{V}/9)

H₅=H₁−(4×l_{V}/9)

ΣH₆₋₁₀=ΣH₁₋₅

However, the closed form solution of the LSI model only exists without absorption, reflection and refraction. Reflection and refraction can be ignored for simplicity of the model, but it is useful to provide some estimation of absorption which can significantly attenuate the radiation. This can be determined as the percent UV transmission per unit length to the power of the average of the distance traveled by the radiation from the center of the lamp and each electrode. The attenuated radiation (Iₐₜₜₙ) is then calculated as follows.

$${I}_{attn} = {\%}_{T}^{{d}_{1}} \times I$$

$${d}_{I} = \frac{{[ {R}^{2} + {( H - l / 2 )}^{2} ]}^{1 / 2} + {[ {R}^{2} + {H}^{2} ]}^{1 / 2} + {[ {R}^{2} + {( H + l / 2 )}^{2} ]}^{1 / 2}}{3}$$

The following tables of fluence (I) and absorption attenuated fluence (Iₐₜₜₙ) are generated with the algorithms as described above.

| | | |
|---|---|---|
| Point | I (W/cm²) | Iₐₜₜₙ (W/cm²) |

Path 1

| P1 | 0.020 | 0.014 |
|---|---|---|
| P2 | 0.033 | 0.026 |
| P3 | 0.068 | 0.056 |
| P4 | 0.157 | 0.137 |
| P5 | 0.234 | 0.210 |
| P6-P10 | | 0.089 (Avg) × 2 |
| Total Average Fluence | | 0.178 |
| Path 2 | | |
| P11 | 0.013 | 0.009 |
| P12 | 0.018 | 0.013 |
| P13 | 0.024 | 0.018 |
| P14 | 0.031 | 0.024 |
| P15 | 0.035 | 0.028 |
| P16-P20 | | 0.018 (Avg) × 2 |

| | |
|---|---|
| Total Average Fluence | 0.036 |

It becomes apparent that the average fluence of 178 mW/cm² in the region between the lamp 12 and the transparent tube 112 is substantially higher than the average fluence between the transparent tube 112 and the wall 128 of the vessel 14 at 36 mW/cm². Much of the region of highest fluence and would have been otherwise occupied by the cooling jacket of the prior-art. Thus, the system 10 is believed to be capable of providing a more effective dose than prior art PUV disinfection systems.

The residence time (t_{R}) is adjusted for percent of plug flow, conservatively estimated at about 60% in this example, and is determined for each path as follows:

$${t}_{R} Path 1 ( sec ) = [ π \times {( \frac{{d}_{T}}{2} )}^{2} - π \times {( \frac{{d}_{L}}{2} )}^{2} ] \times {l}_{V} \times ( \frac{2.642 {E}^{- 4} G}{1 {cm}^{3}} ) / ( {F}_{R} \times \frac{60 sec}{1 min} ) \times {\%}_{PF}$$

$${t}_{R} Path 2 ( sec ) = [ π \times {( \frac{{d}_{V}}{2} )}^{2} - π \times {( \frac{{d}_{T}}{2} )}^{2} ] \times {l}_{V} \times ( \frac{2.642 {E}^{- 4} G}{1 {cm}^{3}} ) / ( {F}_{R} \times \frac{60 sec}{1 min} ) \times {\%}_{PF}$$

The total dose delivered by system 10 is estimated at 164 mJ/cm² and is calculated as follows:
Total Dose (J/cm²)=([Path 1 Avg Fluence (W/cm²)]×[t_{R} Path 1 (sec)])+([Path 2 Avg Fluence (W/cm²)]×[t_{R} Path 2 (sec)]

Although the derived disinfection dose of 164 mJ/cm² is a rough estimate, a more accurate calculation would require intensive computer modeling of fluid dynamics and the complete integration of fluence distribution within the reactor vessel. Additionally, the derived value of the dose is about four times the dose required by the National Sanitation Foundations (NSF) at 40 mJ/cm² to achieve class-A certification, and is produced by system 10 operating at a low frequency of about 5 Hz and an average power of about 30 W representing only about 16% (30 W/180 W) of the estimated power capacity of the system 10, as limited by the cooling method employed by the system 10.

The system 10 has thus been shown to provide significant disinfection results at the power level examined, and the power can be increased substantially if required, thereby demonstrating the viability of the system 10. Moreover, the data suggests the power can actually be decreased and still achieve levels of disinfection results such as those required by the NSF.

For example, the power of the system 10 can be reduced by about 2.5 times to produce a theoretical 65 mJ/cm² dose at about 12 W. Achieving a 65 mJ/cm² dose at 12 W with a mercury-free miniaturized and robust water disinfection device, such as the system 10, is desirable for portable applications because the average power can be supplied by ordinary batteries, and the efficiency provides an ample supply of disinfected water from one battery charge. The energy required per gallon, in this example, is calculated at about 1,440 J:

$$.5 G P M \times \frac{1 min}{60 sec} = 8.333 {E}^{- 3} G P S ( Gallons \text{-} per \text{-} second )$$

$$12 W = \frac{12 J}{sec} or \frac{12 J}{8.333 {E}^{- 3} G P S} = 1 , 440 J \text{/} G$$

An ordinary 12 V, 2 AHr (amp-hour) battery provides about 86,400 J of energy. This battery powering system 10 should provide about 60 gallons of water disinfected with a dose of about 65 mJ/cm² with one charge.

$$12 V , 2 A Hr = 24 W for 1 Hr \times \frac{60 min}{1 Hr} \times \frac{60 sec}{1 min} = 86 , 400 J \times \frac{1 G}{1 , 440 J} = 60 Gallons$$

The power of the system 10, and the corresponding flow rate can be adjusted to the average power that can be supplied by the battery. Additionally, a batch-mode operation would allow for the time interval between pulses to increase as the voltage and current supplied by the battery decreases so that the battery remains useful to the end of its life. This is only possible for liquid disinfection systems, such as system 10, which utilize a pulsed radiation source, because the energy drawn from the battery is stored in the main discharge capacitor 46 before each discharge, and the time required to store the energy can be allowed to increase in batch mode applications. Thus, the germicidal radiation of system 10 remains optimized to the end of battery life, because it is affected only by the discharge event. A constant radiation source, such as a mercury vapor lamp, by contrast, would decline in efficiency towards the end of its battery life.

The system 10 has a ratio of gas discharge arc length to reactor vessel length that is about 35%. The ratio of gas discharge arc length to reactor vessel length is preferably less than about 60% in alternative embodiments. Moreover, the relatively low arc length in relation to the vessel length, and more specifically to the diameter of the bore of the lamp 12, facilitates the utilization of flash-lamps with a Kₒ that is less than about 28 ohm-ampere^{1/2}, and preferably is between about 1 ohm-ampere^{1/2} and about 15 ohm-ampere^{1/2}. Consequently, the lamp 12 requires a relatively low electromotive force to produce an effective current density, and consequently, efficient UV. Additionally, due to its relatively small dimensions, the lamp 12 requires a relatively low amount of stored energy per pulse to achieve an effective power density. These properties can facilitate the use of ordinary, inexpensive, and miniaturized power supply components, and can improve the safety of the system 10. For example, the system 10 can be constructed with substantially the same operating voltages as digital camera photo-flash circuits.

Additionally, the ratio of gas discharge arc length to reactor vessel length of about 35% to about 60% serves to increase the residence time within the reactor vessel of the system 10 without expanding the diameter of the reactor vessel to a point that will create a region of distance-attenuated UV light along the inner surface of the reactor vessel wall through which pathogens may travel receiving an ineffective dose of germicidal radiation.

For example, it is determined using the previously described LSI model and parameters that the maximum fluence without absorption (I), along the second path, composed of points P11 through P20 occurs at the center of the lamp [Path 2 Imax] and has a value of 36 mW/cm². The fluence (I) at point [P11] is calculated to be 13 mW/cm². Therefore, the farthest region from the center of the lamp 12 along the second path is believed to provide about 36% of the maximum disinfection capability of the second path. Moreover, the fluence at point P11 represents about 81% of the maximum fluence at the wall 128 of the vessel 14 calculated at 16 mW/cm².

Thus, the regions behind the electrodes of the lamp 12, created by the relatively low arc length relative to vessel length, are believed to facilitate an effective dose of germicidal radiation and allow the fluid to decelerate or accelerate in the regions 198, 199 so that the flow is more uniform in the regions located nearer the center of the lamp 12, where a more significant portion of the dose is delivered.

A biodosimetry test was performed on system 10, operating at four power levels, with a challenge organism known for its resistance to conventional mercury UV disinfection called MS-2 bacteria phage. The results of the test are as follows.

| SAMPLE | MS-2 PFU/ml in Water |
|---|---|
| Starting Material | 8 × 10⁵ |
| 1 (Device Off) | 2.2 × 10⁵ |
| 2 (20 Hz, 120 W) | <50* |
| 3 (15 Hz, 90 W) | <50* |
| 4 (10 Hz, 60 W) | <50* |
| 5 (5 Hz, 30 W) | <50* |

*0 plaques in 20 ul of neat sample

The results show that there were no viable pathogens in the liquid after the liquid flowed through system 10, at 0.5 GPM, at any of the power levels tested. Although the study shows that the disinfection capability of the device is highly satisfactory, the actual disinfection capability of system 10 cannot be determined by the results. The most that can be determined is the maximum log reduction (from sample 1) that was produced by the lowest power (sample 5) and the equivalent dose relative to conventional LP mercury based systems.

$$Log Inactivation \text{:} Log ( \frac{2.2 {E}^{5}}{1} ) = 5.35 Log$$

The NSF has determined that the log reduction of MS-2 corresponding to their required 40 mJ/cm² dose for class-A certification is about 1.6 to about 2.2, with ˜1.9 being the estimated average. Therefore, the corresponding dose per log inactivation is estimated to be about 21 mJ/cm² per log reduction of MS-2.

$$\frac{40 mJ / {cm}^{2}}{1.9 log} = 21 mJ / {cm}^{2} per log$$

The corresponding dose, relative to LP mercury based systems, delivered by system 10 operating at the lowest power tested is then estimated to be about 112 mJ/cm², or more than about 2.5 times the required dose for class-A certification.

$$5.35 log \times \frac{21 mJ / {cm}^{2}}{1 log} = 112 mJ / {cm}^{2}$$

The testing results also indicate that system 10 could provide a dose of about 259 mJ/cm², as desired by the EPA and the state of California to provide a 4-log reduction of Adenovirus, at a pulse rate of about 12 Hz.

There is, however, a 100 PFU/ml resolution in the assay used to determine the counts of viable MS-2. Factoring in this uncertainty, the results can also be interpreted as follows.

$$Log Inactivation ( with uncertainty ) \text{:} Log ( \frac{2.2 {E}^{5}}{100} ) = 3.25 Log$$

$$Dose ( with uncertainty ) \text{:} 3.25 log \times \frac{21 mJ / {cm}^{2}}{1 log} = 68.25 mJ / {cm}^{2}$$

Factoring the uncertainty of the assay resolution, the dose also exceeds the requirement of NSF class-A certification. However, it is likely the former calculation of about 112 mJ/cm² is the more realistic dose because it more closely matches the theoretical dose and because zero viable pathogens in all samples indicates that the true disinfection level has yet to be probed by the biodosimetry testing.

The system 10 was then challenged with purified water spiked with endotoxin flowing at about 0.5 GPM. The data points, including three power levels, from the assay determining the concentration of endotoxin showing activity before and after treatment is summarized below.

| | ENDOTOXIN μg/ml | | | |
|---|---|---|---|---|
| SAMPLE | TEST 1 | TEST 2 | AVERAGE | STD DEV |
| Starting Material | 2.166 | 3.029 | 2.633 | 0.610 |
| 1 (Device Off) | 2.725 | 2.994 | 2.89 | 0.190 |
| 2 (20 Hz, 120 W) | 1.502 | 1.527 | 1.54 | 0.017 |
| 3 (15 Hz, 90 W) | 1.747 | 1.631 | 1.72 | 0.082 |
| 4 (10 Hz, 60 W) | 1.782 | 1.806 | 1.82 | 0.017 |
| 5 (5 Hz, 30 W) | | | *1.97 | |

*—Extrapolated (y = mx + b) from samples 2-4
$Conversion \text{:} μ g / ml \times \frac{\text{1,000} ng}{1 μ g} \times \frac{10 EU}{1 ng} = EU / ml$

The concentration of the pass through sample (sample 1) is about 2.89 μg/ml or about 28,900 EU/ml. The corresponding endotoxin reduction for each sample is summarized as follows.

| | |
|---|---|
| Sample 2 = 1.54 μg/ml | Reduction = 1.35 μg/ml (13,500 EU/ml) |
| Sample 3 = 1.72 μg/ml | Reduction = 1.17 μg/ml (11,700 EU/ml) |
| Sample 4 = 1.82 μg/ml | Reduction = 1.07 μg/ml (10,700 EU/ml) |
| Sample 5 = 1.97 μg/ml | Reduction = 0.92 μg/ml (9,200 EU/ml) |

Excluding the extrapolated data point (sample 5), the data corresponds to an average endotoxin unit inactivation per mille-liter per watt value for system 10 of about 140.

$$\frac{\begin{matrix}( \frac{13 \text{,} 500 EU / ml}{120 W} ) + ( \frac{11 \text{,} 700 EU / ml}{90 W} ) + \\ ( \frac{10 \text{,} 700 EU / ml}{60 W} )\end{matrix}}{3} = 140 EU / ml / W$$

The efficiency of pathogen and corresponding pyrogen inactivation, illustrated by the above data, and the instantaneous nature of system 10 would be beneficial to water-for-injection applications such as pharmaceutical process equipment at points-of-use and medical devices such as dialysis equipment.

A comparison of endotoxin inactivation efficiency of the system 10 to a study performed with MP mercury vapor lamp technology published the journal of “Applied and Environmental Microbiology” in May of 2003 entitled “Endotoxin Inactivation in Water by Using Medium-Pressure Lamps” reveals that the system 10 is significantly more effective.

A table of the theoretical endotoxin inactivation after UV exposure from the publication is included for reference below.

| | Theoretical endotoxin concn (EU/ml) after | | | | | |
|---|---|---|---|---|---|---|
| | UV irradiation of samples with an | | | | | |
| UV fluence | initial endotoxin concn (EU/ml) of: | | | | | |
| (mJ/cm²) | 10 | 25 | 50 | 100 | 150 | 200 |
| 40 | 0 | 3 | 28 | 78 | 128 | 178 |
| 60 | 0 | 0 | 17 | 67 | 117 | 167 |
| 80 | 0 | 0 | 6 | 56 | 106 | 156 |
| 100 | 0 | 0 | 0 | 45 | 95 | 145 |

The reason that the data from the table is substantially uniform is that all the concentrations are theoretical and calculated from an EU/ml reduction of about 0.55 for each mJ/cm² of dose.

The EU/ml reduction for each mJ/cm² of dose of the system 10 is estimated from the previous data to be about 82 EU/ml per mJ/cm² dose or about 135 EU/ml per mJ/cm² when factoring in the uncertainty of the PFU resolution.

$$\frac{9 \text{,} 200 EU / ml}{112 mJ / {cm}^{2}} = 82 EU / ml per mJ / {cm}^{2} Dose$$

$$\begin{matrix}\frac{9 \text{,} 200 EU / ml}{68 mJ / {cm}^{2}} = 135 EU / ml per mJ / {cm}^{2} Dose \\ ( with MS2 uncertainty )\end{matrix}$$

In this case, system 10 is between about 149 to about 245 times more effective on endotoxin than the medium pressure mercury vapor lamp of the cited study. However, the EU reduction per mJ/cm² dose of system 10 is likely to be decreased as an increase in MS-2 dose efficiency is proven in subsequent biodosimetry testing.

Additionally, the same study cites that from several sources that the typical concentration of endotoxin in untreated water ranges from about 1 to about 400 EU/ml. Therefore, system 10 should provide insurance of substantially zero endotoxin activity in a water-for-injection or dialysis applications with a NSF disinfection level treatment.

An estimated lifetime of the lamp 12 of the system 10 as a projected number of shots can be calculated by the ratio of the operating energy to the explosion energy as follows.

$$u - Explosion Energy ( J ) = {kdlt}_{1 / 3}^{\frac{1}{2}} = 90 \times 4 \times ( 2 / 2.54 ) \times {.028}^{\frac{1}{2}} = 47$$

k—Constant, 90

d—Diameter of lamp envelope inner bore in mm

l—Arc length of lamp in inches

t_{1/3}—Pulse Width in ms

$$S - Projected Life ( shots ) = {( \frac{6 J}{47 J} )}^{- 8.5} = 3.97 {E}^{7}$$

Due to the fact that the system 10 does not employ a simmer circuit to extend the lifetime of the lamp 12, and because the above calculations are generally used to determine the life of flash-lamps for use with visible spectra rather than UV which is more easily attenuated than visible radiation, the projected life is discounted by 70% as follows:

$$3.97 {E}^{7} shots \times 70 \% = 2.78 {E}^{7} Shots$$

$$\frac{2.78 {E}^{7 } Shots }{5 Hz} \times \frac{1 min}{60 sec} = \frac{92 \text{,} 667 min}{10 min perday } = 9 \text{,} 267 days$$

The system 10 can potentially operate for 10 minutes per day at a 0.5 GPM, providing 5 gallons of disinfected water per day for 9,267 days or 25 years. This estimate represents 64 days or 2 months of continuous operation, and illustrates that the intermittent operation of the system 10 and low frequency pulse rate facilitated by the elimination of the simmer circuit of the prior art, can prolong the maintenance period of the lamp 12 significantly.

The fluence delivered by the system 10 can be adjusted to compensate for aging of the lamp 12 by modulation of the pulse frequency by the power supply from stored empirical data. For example, a life cycle test can be performed on the lamp 12 and can show, for example, that the germicidal radiation attenuates to an unacceptable level of about 70% after 27.8 million shots. The microcontroller 28 can compensate for the linear attenuation of the lamp 12 and deliver a consistent dose to the end of lamp life by implementing the following algorithm.

$$Mod Hz = \frac{Input Hz}{1 - [ \frac{Shot Count}{Life Shots} ] \times Attn F}$$

The Input Hz is selected by the user for the device's application. The Shot Count is the current number of shots delivered by the power supply since the last lamp replacement. The Life Shots is the number of shots that the lamp produced at the point where the Attn F, attenuation factor, was measured. In this example the Life Shots is 27.8 million and the Attn F is 70%. The variables Life Shots and Attn F are stored constants in the microcontroller and the variable Shot Count is incremented with each pulse.

The pulse duration, tₚᵤₗₛₑ, is always constant and is determined mainly as the time required to extinguish the lamp from the DC/DC converter or TRIAC. For the case of a 20 kHz oscillator, for example, a four-cycle time-out is 200 μs. Therefore, the modulation of the lamp power is accomplished by modifying the Time-Out value which is the time duration between the end of the last pulse and the initiation of the next. The modified Hz value is used to determine the time between pulses as follows.
Time Out=Mod Hz⁻¹=tₚᵤₗₛₑ

The foregoing description is provided for the purpose of explanation and is not to be construed as limiting the invention. Although the invention has been described with reference to preferred embodiments or preferred methods, it is understood that the words which have been used herein are words of description and illustration, rather than words of limitation. Furthermore, although the invention has been described herein with reference to particular structure, methods, and embodiments, the invention is not intended to be limited to the particulars disclosed herein, as the invention extends to all structures, methods and uses that are within the scope of the appended claims. Those skilled in the relevant art, having the benefit of the teachings of this specification, may effect numerous modifications to the invention as described herein, and changes may be made without departing from the scope and spirit of the invention as defined by the appended claims.

What is claimed is:

1. A system for disinfecting a fluid, comprising:
a substantially transparent tube;
a source of mercury-free ultraviolet light positioned substantially within the tube; and
a vessel defining a volume, wherein the tube is positioned substantially within the volume and has a first open end in fluid communication with the volume, the vessel has a first port formed therein, and the first port is in fluid communication with a second open end of the tube so that the tube and the source of mercury-free ultraviolet light form a flow path for the fluid.

2. The system of claim 1, wherein the vessel has a second port in fluid communication with the volume so that the volume further defines the flow path for the fluid.

3. The system of claim 1, wherein the source of ultraviolet light is a flash-lamp.

4. The system of claim 1, wherein the tube is formed from quartz.

5. The system of claim 2, wherein the fluid flows through the tube in a first direction, and the fluid flows through the volume in a second direction opposite the first direction.

6. The system of claim 3, further comprising an external trigger circuit.

7. The system of claim 6, wherein the electrical discharge is initiated exclusively by the external trigger circuit.

8. The system of claim 3, wherein the flash-lamp has a lamp-resistance parameter (K₀) no greater than about 28 ohm-ampere^{1/2}.

9. The system of claim 8, wherein the lamp-resistance parameter is between about 1.0 ohm-ampere^{1/2} and about 15 ohm-ampere^{1/2}.

10. The system of claim 3, wherein the flash-lamp extends substantially in a first direction, and an arc length of the flash-lamp is about thirty-five percent to about sixty percent of a length of the volume in the first direction.

11. The system of claim 3, wherein the flash-lamp comprises a first and a second electrode, the flash-lamp extends substantially in a first direction, and a length of each of the first and second electrodes is at least about seventeen percent of a length of the volume in the first direction.

12. The system of claim 1, wherein the vessel comprises means for dividing a flow of the fluid into at least two streams of substantially equal flow rate and flow volume and directing the at least two streams to the flow path formed by the tube and the source of mercury-free ultraviolet light.

13. The system of claim 2, wherein the vessel comprises means for combining at least two streams of the fluid of substantially equal flow rate and flow volume into a single flow after the fluid has traveled through the volume.

14. The system of claim 1, wherein the vessel defines at least two internal flow passages of substantially equal dimensions for directing the fluid to the volume.

15. The system of claim 14, wherein the internal flow passages are defined by a channel formed in the vessel.

16. The system of claim 15, wherein a first of the internal flow passages is further defined by the first port, and a second of the internal flow passages is further defined by a second port formed in the vessel and in fluid communication with the second open end of the tube.

17. The system of claim 16, wherein the channel divides a flow of the fluid into two streams of substantially equal flow rate and flow volume.

18. The system of claim 16, wherein the vessel defines four of the internal flow passages and the four internal flow passages are defined by two of the channels.

19. The system of claim 18, wherein the two channels divide two flows of the fluid into four streams of substantially equal flow rate and flow volume.

20. The system of claim 19, wherein a third of the internal flow passages is further defined by a third port formed in the vessel, a fourth of the internal flow passages is further defined by a fourth port formed in the vessel, and the four streams enter the flow path defined by the tube and the source of mercury-free ultraviolet light respectively by way of the first, second, third, and fourth ports.

21. The system of claim 18, further comprising a fitting that divides a stream of the fluid into two streams of substantially equal flow rate and flow volume, wherein a first of the streams is directed to a first of the channels, and a second of the streams is directed to a second of the channels.

22. The system of claim 17, wherein the channel is substantially Y-shaped.

23. The system of claim 21, wherein the vessel further comprises a top portion, a mid portion having the channels and the first, second, third, and fourth portions formed therein, and a bottom portion.

24. The system of claim 23, wherein an inwardly-facing circumferential surface of the bottom portion and an inwardly-facing surface of the mid portions define the volume.

25. The system of claim 23, wherein the top portion has two ports formed therein that respectively receive the two streams of the fluid.

26. The system of claim 2, wherein the vessel defines at least two internal flow passages of substantially equal dimensions for directing the fluid from the volume.

27. The system of claim 26, wherein the internal flow passages are defined by a channel formed in the vessel.

28. The system of claim 26, wherein a first of the internal flow passages is further defined by the second port, and a second of the internal flow passages is further defined by a third port formed in the vessel and in fluid communication with the volume.

29. The system of claim 28, wherein the channel divides a flow of the fluid into two substantially equal streams.

30. The system of claim 28, wherein the vessel defines four of the internal flow passages and the four internal flow passages are defined by two of the channels.

31. The system of claim 30, wherein the two channels channel combine four flows of the fluid into two streams of substantially equal flow rate and flow volume.

32. The system of claim 31, wherein a third of the internal flow passages is further defined by a fourth port formed in the vessel, a fourth of the internal flow passages is further defined by a fifth port formed in the vessel, and the four substantially equal streams enter the flow path defined by the tube and the source of mercury-free ultraviolet light respectively by way of the second, third, fourth, and fifth ports.

33. The system of claim 30, further comprising a fitting that combines the two streams into a single flow.

34. The system of claim 27, wherein the channel is substantially arc-shaped.

35. The system of claim 33, wherein the vessel further comprises a top portion, a mid portion having the channels and the first, second, third, and fourth portions formed therein, and a bottom portion.

36. The system of claim 35, wherein the top portion has two ports formed therein that respectively receive the two substantially equal streams of the fluid.

37. The system of claim 11, wherein at least a portion of each of the first and second electrodes is encapsulated in a material is selected from the group consisting of glass and quartz.

38. The system of claim 2, further comprising:
a first valve located upstream of and proximate to the first port, the first valve preventing flow of the fluid toward the first port on a selective basis; or
a second valve located downstream of and proximate to the second port, the second valve preventing flow of the fluid from the second port on a selective basis.

39. The system of claim 6, wherein the external trigger circuit comprises a transformer, and an electrical conductor electrically connected to a first winding of the transformer and located proximate the flash-lamp.

40. The system of claim 39, wherein energization of the first winding generates a high-voltage pulse that causes gas inside the flash-lamp to ionize.

41. The system of claim 40, wherein the external trigger circuit further comprises a first capacitor electrically connected to a second winding of the transformer, a thyristor, and an opto-isolated random-phase TRIAC driver that connects the potential of first capacitor to a gate of a trigger thyristor so that current from the first capacitor is directed through the second winding of transformer.

42. The system of claim 41, further comprising a second capacitor electrically connected to the flash lamp.

43. The system of claim 42, further comprising a source of electrical potential electrically connected to the first and second capacitors.

44. The system of claim 43, wherein the source of electrical potential is a direct-current (DC) power source.

45. The system of claim 44, further comprising a DC inverter electrically connected to the DC inverter, and a voltage multiplier electrically connected to the DC inverter and the first and second capacitors.

46. The system of claim 44, wherein the ionization of the gas inside the flash-lamp causes the gas to conducts current from the second main discharge capacitor though the lamp.

47. The system of claim 41, further comprising a signal processor, wherein an initial pulse of the flash-lamp is initiated by sending a signal from the signal processor the TRIAC driver.

48. The system of claim 47, wherein the signal processor is a microcontroller; a SIDAC; an RC circuit comprising the first capacitor and a potentiometer, or a timing device.

49. The system of claim 47, further comprising setting switches or a rheostat electrically connected to the signal processor for varying a pulse rate of the flash-lamp.

50. The system of claim 47, further comprising a resistor, and one of an opto-isolated depletion-mode field-effect transistor and a relay electrically connected to the resistor and communicatively coupled to the signal processor for discharging the first and second capacitors on a selective basis.

51. The system of claim 45, further comprising a first field-effect transistor for timing out a gate of a second field effect transistor on a gate of a high-frequency oscillator of the DC inverter.

52. The system of claim 51, wherein the first field effect transistor is an opto-isolated.

53. The system of claim 47, wherein the signal processor is configured to vary a rate at which pulses of the pulsed ultraviolet light are generated in response to aging of the flash-lamp.

54. The system of claim 47, wherein the signal processor is configured to vary the dose of ultraviolet light delivered to the fluid.

55. The system of claim 47, wherein the signal processor is configured to pulse the flash-lamp at least once after ceasing flow of the fluid through the flow path.

56. The system of claim 47, wherein the external trigger circuit further comprises a resistor electrically coupled to the first capacitor on a selective basis, and an opto-isolated, depletion-mode field-effect transistor that electrically connects the first capacitor and the resistor in response to a signal from the signal processor indicating that the flow of the fluid through the first and second flow paths has ceased.

57. The system of claim 3, wherein the flash-lamp has a fill pressure between about 100 torr and about 650 torr.

58. The system of claim 3, wherein the flash-lamp comprises a first and a second electrode, the flash-lamp defines a volume for holding a gas, and the flash-lamp has a tipoff located behind one of the first and second electrodes in relation to the volume.

59. A system for disinfecting a fluid, comprising:
a vessel defining a volume;
a flash-lamp positioned substantially within the volume; and
an external trigger circuit that exclusively initiates an electrical discharge in the flash-lamp.

60. The system of claim 59, further comprising a substantially transparent tube, the flash-lamp being positioned substantially within the tube.

61. The system of claim 60, wherein the tube, the flash-lamp, and the volume define a flow path for the fluid.

62. A process for disinfecting a fluid, comprising:
flowing the fluid through a first flow path defined by a mercury-free source of ultraviolet light and an inner surface of a substantially transparent tube;
irradiating the fluid with the mercury-free source of ultraviolet light as the fluid flows through the first flow path;
flowing the fluid through a second flow path defined by an outer surface of the substantially transparent tube; and
irradiating the fluid with the mercury-free source of ultraviolet light as the fluid flows through the second flow path.

63. The process of claim 62, wherein the second flow path is further defined by a perimeter of a volume that holds the mercury-free source of ultraviolet light and the substantially transparent tube.

64. The process of claim 62, wherein the mercury-free source of ultraviolet light is a flash-lamp.

65. The process of claim 64, further comprising initiating an electrical discharge in the flash-lamp using only an external trigger circuit.

66. The process of claim 62, wherein irradiating the fluid with the mercury-free source of ultraviolet light as the fluid flows through the second flow path comprises irradiating the fluid with the mercury-free source of ultraviolet light through the substantially transparent tube.

67. The process of claim 62, wherein:
flowing the fluid through a first flow path defined by a mercury-free source of ultraviolet light and an inner surface of a substantially transparent tube comprises flowing the fluid substantially in a first direction; and
flowing the fluid through a second flow path defined in part by an outer surface of the substantially transparent tube comprises flowing the fluid substantially in a second direction opposite the first direction.

68. The process of claim 62, further comprising splitting the fluid into a plurality of streams each having a substantially equal flow rate and flow velocity before flowing the fluid through the first and second flow paths.

69. The process of claim 62, further comprising splitting the fluid into a plurality of streams each having a substantially equal flow rate and flow velocity after flowing the fluid through the first and second flow paths.

70. The process of claim 62, further comprising:
activating the mercury-free source of ultraviolet light in response to an electrical signal indicating that the flow of the fluid toward the first and second flow paths has been initiated; and
deactivating the mercury-free source of ultraviolet light in response to another electrical signal indicating that the flow of the fluid through the first and second flow paths has ceased.

71. The process of claim 62, further comprising ceasing the flow of the water through the first and second flow paths and pulsing the mercury-free source of ultraviolet light at least once after ceasing the flow of the water through the first and second flow paths.

72. The process of claim 62, further comprising varying an average power of the mercury-free source of ultraviolet light in response to variations in a flow rate of the water through the first and second flow paths.

73. The process of claim 62, further comprising varying a rate at which pulses of the flash lamp are generated in response to variations in a flow rate of the water through the first and second flow paths.

74. A process for eliminating endotoxins from a fluid, comprising: irradiating the fluid with a mercury-free source of ultraviolet radiation in doses of about 1 mJ/cm² to about 300 mJ/cm².

75. The process of claim 74, wherein the mercury-free source of ultraviolet radiation is a flash-lamp.
